(19) United States
(12) Patent Application Publication
(10) Pub. No.: US 2023/0157785 A1
(43) Pub. Date: May 25, 2023

(54) MOTION BASED DENTAL SPLINTS

(71) Applicant: VisionX, LLC, Edina, MN (US)

(72) Inventors: Michael C. Marshall, Edina, MN (US); Bruce Willard Hultgren, Victoria, MN (US)

(21) Appl. No.: 17/919,385

(22) PCT Filed: Apr. 16, 2021
(86) PCT No.: PCT/US2021/027760

§ 371 Date: Oct. 17, 2022

Related U.S. Application Data
(60) Provisional application No. 63/010,821, filed on Apr. 16, 2020.

(51) Int. Cl. A61C 5/00; A61C 9/00

(52) U.S. Cl. CPC A61C 5/007; A61C 9/0053

(57) ABSTRACT

Apparatuses, components, devices, methods, and systems for generating motion-based dental splints are provided. An example dental splint includes a thin-shell aligner and a contact surface formed based on motion data. The motion data may include relative motion data based on movement of a patients upper dentition with respect to the patients lower dentition. The contact surface may include one or more ridges corresponding to the positions of a contact region on the opposing dentition as a jaw movement is performed. The dental splint may be used to treat temporomandibular joint disorder. An example method includes acquiring an impression of a patients dentition; acquiring jaw motion data for the patient; generating a thin-shell appliance for the patient based on the impression; generating a contact surface based on the motion data; and joining the contact surface with the thin-shell appliance.

### CROSS-REFERENCE TO RELATED APPLICATIONS

This application claims priority, as appropriate, to U.S. Ser. No. 63/010,821, titled “MOTION BASED DENTAL SPLINTS,” and filed Apr. 16, 2020, the disclosure of which is hereby incorporated by reference in its entirety.

### BACKGROUND

Understanding and recording an accurate static relationship between teeth in a patient's upper jaw and lower jaw is an important first step in the art and science of designing dental appliances or restorations and planning dental or surgical interventions that affect dental/skeletal function and aesthetics of the facial musculature system.

Additionally, the dynamic motion of the lower jaw and dentition interacting functionally and aesthetically is even more important in the various reconstructive domains in dentistry and medicine that require precise knowledge and locations of the musculoskeletal-dental components that define this motion. The greater accuracy of motion definition allows for more precise design of restorations (e.g., crowns, implants, full/partial prosthesis) and associated macro procedures such as orthognathic surgery, trauma reconstruction, etc. These physical components can be described in engineering terms as a kinematic linkage system incorporating the relationship of the temporomandibular joint to the dentition and soft tissue of the face. This linkage definition has only been approximated poorly by traditional articulator devices and systems in dentistry.

Dental appliances may be used in the treatment of various dental conditions. Examples of dental appliances include therapeutic appliances and restorative appliances (dental restorations). Non-limiting examples of therapeutic appliances include surgical splints, therapeutic splints, occlusal splints, orthodontic retainers, and orthodontic aligners. An example of a therapeutic splint is a splint for the treatment of temporomandibular joint disorders (TMD), which may be referred to as a TMD splint. Another example of a therapeutic splint is a splint for the treatment of sleep apnea, which may be referred to as a sleep apnea splint.

A dental restoration is a type of dental appliance that is used to restore a tooth or multiple teeth. For example, a crown is a dental restoration that is used to restore a single tooth. A bridge is another example of a dental restoration. A bridge may be used to restore one or more teeth. A denture is another example of a dental restoration. A denture can be a full or partial denture. Dentures can also be fixed or removable. An implant is yet another example of a dental restoration. Dental implants are prosthetic devices that are placed in bone tissue of a patient's jaw and used to secure other dental restorations such as implant abutments and crowns, or partial and full dentures. In some circumstances, dental restorations are used to restore functionality after a tooth is damaged. In other circumstances, dental restorations are used to aesthetically improve a patient's dentition.

When complex or multiple dental appliances, dental restorations, or dental therapies are applied to a patient simultaneously, errors or inaccuracies in the representation of dental motion are compounded, resulting in inadequate or suboptimal results for patients. In the worst case, inaccurate motion data can result in the complete failure of the appliances, restorations, or treatment at very high cost clinically, financially, and emotionally.

Jaw and facial movement may be determined by attaching a device to the patient's dentition.

### SUMMARY

In general terms, this disclosure is directed to motion-based dental splints and systems for generating motion-based dental splints. In one possible configuration and by non-limiting example, a patient assembly is coupled to a patient's dentition and an imaging system captures images of the patient assembly as the patient's dentition moves.

One aspect is a dental splint comprising a thin-shell aligner and a contact surface. The contact surface may be formed based on motion data. In some implementations, the motion data includes relative motion data on movement of a patient's upper dentition with respect to the patient's lower dentition. The contact surface may include one or more ridges corresponding to the positions of a contact region on the opposing dentition as a jaw movement is performed. In some examples, the dental splint is used for treatment of temporomandibular joint disorder.

Another aspect is a method comprising: acquiring an impression of a patient's dentition; acquiring jaw motion data for the patient; generating a thin-shell appliance for the patient based on the impression; generating a contact surface based on the motion data; and joining the contact surface with the thin-shell appliance.

Yet another aspect is a system for forming motion-based dental splints. The system may include a dental impressioning station configured to capture an impression of a patient's dentition. The system may also include a motion capture system configured to capture motion of the patient's dentition. The system may also include a dental splint design system configured to design a motion-based dental splint using the impression of the patient's dentition and the captured motion.

Examples are implemented as a computer process, a computing system, or as an article of manufacture such as a device, computer program product, or computer readable medium. According to an aspect, the computer program product is a computer storage medium readable by a computer system and encoding a computer program comprising instructions for executing a computer process.

The details of one or more aspects are set forth in the accompanying drawings and description below. Other features and advantages will be apparent from a reading of the following detailed description and a review of the associated drawings. It is to be understood that the following detailed description is explanatory only and is not restrictive of the claims.

### BRIEF DESCRIPTION OF THE DRAWINGS

FIG. 1 is a schematic block diagram illustrating an example motion capture system for capturing jaw movement.

FIG. 2 is a block diagram of an example patient assembly of FIG. 1.

FIG. 3 is an example embodiment of the clutch of FIG. 2.

FIG. 4 is a schematic diagram of an example motion capture system of FIG. 1 in which two screens are used.

FIG. 5 illustrates a top view of an embodiment of a reference structure and an embodiment of the imaging system of FIG. 4.

FIG. 6 is a perspective view of the reference structure of FIG. 4 disposed between the screens of the imaging system of FIG. 4.

FIG. 7 is a schematic diagram of another example motion-based dental splint.

FIG. 8 is a schematic diagram of another example motion-based dental splint.

FIG. 9 is a schematic diagram of another example motion-based dental splint.

FIG. 10 is a schematic diagram of another example motion-based dental splint.

FIG. 11 is a schematic diagram of another example motion-based dental splint.

FIG. 12 is a schematic diagram of another example motion-based dental splint.

FIG. 13 is a schematic diagram of another example motion-based dental splint.

FIG. 14 is a schematic diagram of another example motion-based dental splint.

FIG. 15 is a schematic block diagram illustrating an example of a system for using jaw motion captured by the system of FIG. 1 to fabricate a motion-based dental splint.

FIG. 16 is a flowchart of an example method for generating motion-based dental splints.

FIG. 17 illustrates an example architecture of a computing device, which can be used to implement aspects according to the present disclosure.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The present disclosure relates to a motion-based dental splint and a system for generating motion-based dental splints. For example, the motion-based dental splints may include a dentition-coupling region and a contact surface that is generated based on motion data about the patient's jaw. The motion data may be captured with a jaw movement measurement system. For example, the jaw movement measurement system may record the motion of a patient's mandible relative to the patient's maxilla. The motion data may, for example, correspond to motion while the patient's jaw performs various movements such as a protrusive jaw movement (front-to-back) or an excursive movement (side-to-side).

In some embodiments, the jaw movement measurement system infers the approximate location of a screw axis corresponding to the condyloid process of the temporomandibular joint of the patient. Further, the system may generate a model of a range of motion of the mandible relative to the maxilla based on the inferred location of the screw axis, the recorded motion, or both. In some implementations, the motion data may be based on actual recorded motion, a generated model of the range of motion, or both.

In some embodiments, the contact surface is shaped so that when the motion-based dental splint is worn, some or all of the patient's opposing dentition make contact with the contact surface throughout a range of motion. As an example, the contact surface may be shaped so that the patient's opposing cuspids (also referred to as canines) make contact with the contact surface throughout a protrusive jaw movement. As another example, the contact surface may be shaped so that the patient's opposing anterior teeth make contact with the contact surface through an excursive jaw movement. The contact surface may balance the contact force evenly across all of the teeth that are in contact, reducing stress or tension on the temporomandibular joint.

The dentition-coupling region is a region of the splint that is configured to couple the splint to the patient's dentition. For example, the dentition-coupling region may include a thin-shell aligner formed from an impression of the patient's dentition. The thin shell may, for example, follow the contours of at least a portion of the patient's dentition. The thin shell may extend over the height of contours of at least some of the patient's teeth so as to clasp the patient's dentition. In some embodiments, the dentition-coupling region is configured to extend over the patient's posterior teeth so as to prevent super eruption of those posterior teeth when they are held out of contact with the opposing dentition by the contact surface.

In embodiments, motion recorded by the jaw movement measurement system is applied to a three-dimensional digital model of at least a portion of the patient's dentition. This motion can then be used to generate the contact surface. For example, the location of desired contact points on the surface of the opposing dentition may be swept through a specific jaw movement. This path generated by sweeping the contact may define a contact region of the contact surface. Contact points on multiple teeth may be swept through the same jaw movement to define multiple contact regions of the contact surface. These contact regions may be joined together to form the contact surface. In some embodiments, the contact regions are joined by a surface that is offset back from the contact regions so as not to inadvertently interfere with the opposing dentition making contact with the contact regions when the splint is worn.

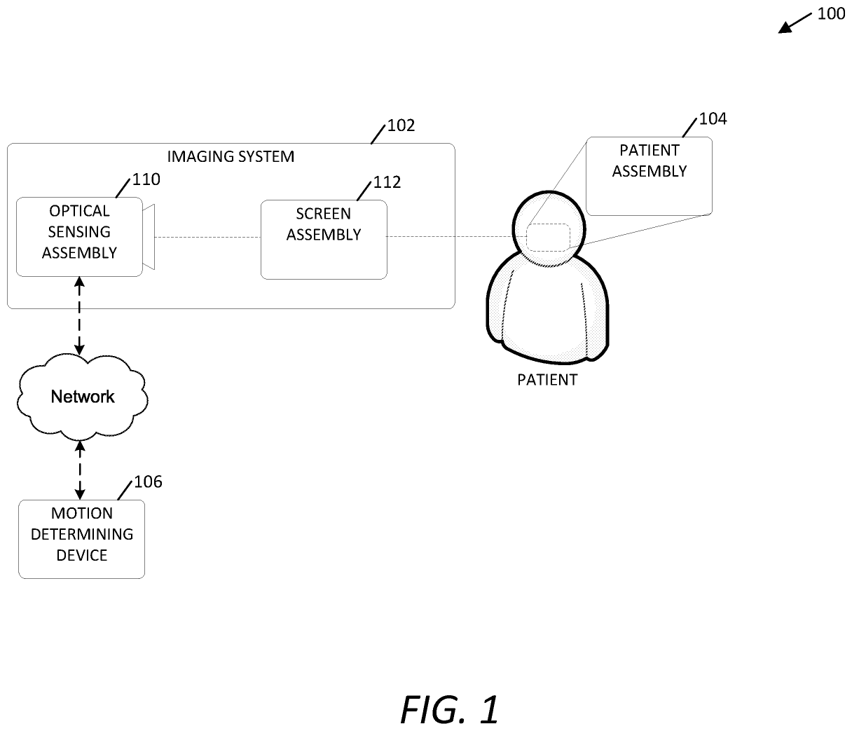

FIG. 1 is a schematic block diagram illustrating an example motion capture system 100 for capturing jaw movement. For example, this motion capture system 100 may capture motion data that can be used to generate motion-based dental splints. It should be understood, however, that other methods of capture motion data may be used to generate some implementations of the dental splint.

In this example, the motion capture system 100 includes an imaging system 102, a patient assembly 104, and a motion determining device 106. Also shown in FIG. 1 are a patient and a network.

In some embodiments, the imaging system 102 includes an optical sensing assembly 110 and a screen assembly 112. The optical sensing assembly 110 may capture a plurality of images as the patient's jaw moves. For example, the optical sensing assembly 110 may include one or more cameras such as video cameras. In some embodiments, the optical sensing assembly 110 captures a plurality of images that do not necessarily include the patient assembly, but can be used to determine the position of the patient assembly 104. For example, the patient assembly 104 may emit lights that project onto surfaces of the screen assembly 112 and the optical sensing assembly 110 may capture images of those surfaces of the screen assembly 112. In some implementations, the optical sensing assembly 110 does not capture images but otherwise determines the position of the projected light or lights on the surfaces of the screen assembly 112.

The screen assembly 112 may include one or more screens. A screen may include any type of surface upon which light may be projected. Some implementations include flat screens that have a planar surface. Some implementations may include rounded screens, having cylindrical (or partially cylindrical) surfaces. The screens may be formed from a translucent material. For example, the locations of the lights projected on the screens of the screen assembly 112 may be visible from a side of the screens opposite the patient assembly 104 (e.g., the screen assembly 112 may be positioned between the optical sensing assembly 110 and the patient assembly 104).

In addition to capturing the images, the imaging system 102 may capture or generate various information about the images. As an example, the imaging system 102 can generate timing information about the images. Although alternatives are possible, the timing information can include a timestamp for each of the images. Additionally, a frame rate (e.g., 10 frames/second, 24 frames/second, 60 frames/second) may be stored with a group of images. Other types of information that can be generated for the images includes an identifier of a camera, a position of a camera, or settings used when capturing the image.

The patient assembly 104 is an assembly that is configured to be secured to the patient. The patient assembly 104 or parts thereof may be worn by the patient and may move freely with the patient (i.e., at least a part of the patient assembly 104 may, when mounted to the patient, move in concert with patient head movement). In contrast, in at least some implementations, the imaging system 102 is not mounted to the patient and does not move in concert with patient head movement.

In some embodiments, the patient assembly 104 may include light emitters that emit a pattern of light that projects on one or more surfaces (e.g., screens of the screen assembly 112), which can be imaged to determine the position of the patient assembly 104. For example, the light emitters may emit beams of substantially collimated light (e.g., laser beams) that project onto the surfaces as points. Based on the locations of these points on the surfaces, a coordinate system can be determined for the patient assembly 104, which can then be used to determine a position and orientation of the patient assembly 104 and the patient's dentition.

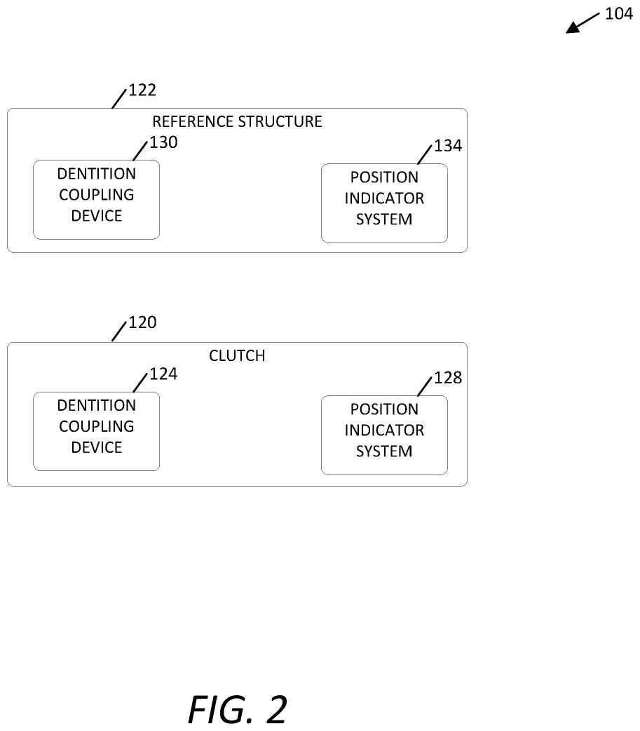

In some embodiments, the patient assembly 104 includes separate components that are configured to be worn on the upper dentition and the lower dentition and to move independently of each other so that the motion of the lower dentition relative to the upper dentition can be determined. Examples of the patient assembly 104 are illustrated and described throughout, including in FIG. 2.

The motion determining device 106 determines the motion of the patient assembly 104 based on images captured by the imaging system 102. In some embodiments, the motion determining device 106 includes a computing device that uses image processing techniques to determine three-dimensional coordinates of the patient assembly 104 (or portions of the patient assembly) as the patient's jaw is in different positions. For example, images captured by the optical sensing assembly 110 of screens of the screen assembly 112 may be processed to determine the positions on the screens at which light from the patient assembly is projected. These positions on the screens of the screen assembly 112 may be converted to three-dimensional coordinates with respect to the screen assembly 112. From those three-dimensional coordinates, one or more positions and orientations of the patient assembly 104 (or components of the patient assembly 104) may be determined.

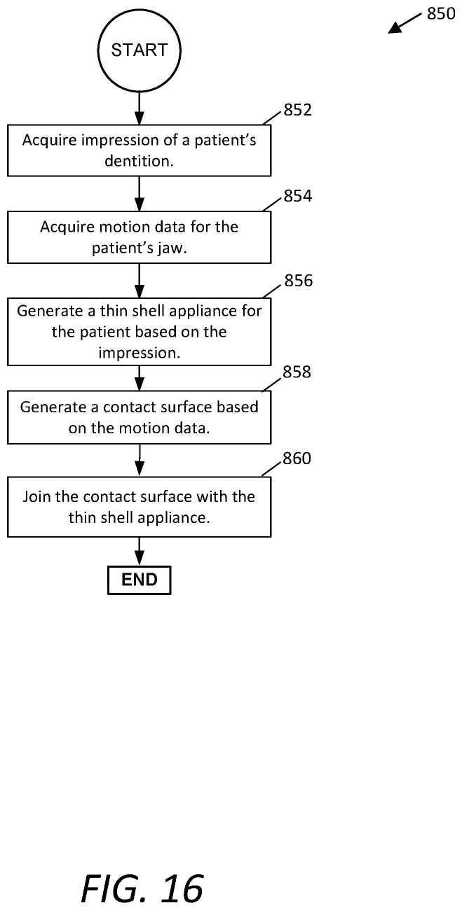

Based on the determined positions and orientations of the patient assembly 104, some embodiments determine the relative positions and movements of the patient's upper and lower dentition. Further, some embodiments infer the location of a kinematically derived screw axis that is usable in modeling the motion of the patient's mandible (including the lower dentition) about the temporomandibular joint. Examples of the motion determining device 106 and operations it performs are illustrated and described throughout, including in FIGS. 16, 17, 28, and 29.

FIG. 2 is a schematic block diagram of an example patient assembly 104. In this example, the patient assembly includes a clutch 120 and a reference structure 122. Here, the clutch 120 and the reference structure 122 are not physically connected and can move independently of one another.

The clutch 120 is a device that is configured to couple to a patient's dentition. For example, the clutch 120 may grip the teeth of the dentition of the patient. In some embodiments, the clutch 120 comprises a dentition coupling device 124 and a position indicator system 128. In some embodiments, the clutch 120 is configured to couple to the lower dentition of the patient so as to move with the patient's mandible. In other embodiments, the clutch 120 may be configured to couple to the patient's upper dentition so as to move with the patient's maxilla.

The dentition coupling device 124 is configured to removably couple to the patient's dentition. In some embodiments, the dentition coupling device 124 rigidly couples to the patient's dentition such that while coupled, the movement of the dentition coupling device 124 relative to the patient's dentition is minimized. Various embodiments include various coupling mechanisms.

For example, some embodiments couple to the patient's dentition using brackets that are adhered to the patient's teeth with a dental or orthodontic adhesive. As another example, some embodiments couple to the patient's dentition using an impression material. For example, some embodiments of the dentition coupling device 124 comprise an impression tray and an impression material such as polyvinyl siloxane. To couple the dentition coupling device 124 to the patient's dentition, the impression tray is filled with impression material and then placed over the patient's dentition. As the impression material hardens, the dentition coupling device 124 couples to the patient's dentition.

Alternatively, some embodiments comprise a dentition coupling device 124 that is custom designed for a patient based on a three-dimensional model of the patient's dentition. For example, the dentition coupling device 124 may be formed using a rapid fabrication machine. One example of a rapid fabrication machine is a three-dimensional printer, such as the PROJET® line of printers from 3D Systems, Inc. of Rock Hill, S.C. Another example of a rapid fabrication machine is a milling device, such as a computer numerically controlled (CNC) milling device. In these embodiments, the dentition coupling device 124 may comprise various mechanical retention devices such as clasps that are configured to fit in an undercut region of the patient's dentition.

Embodiments of the dentition coupling device 124 may be operable to couple to the patient's dentition using a combination of one or more mechanical retention structures, adhesives, and impression materials. For example, the dentition coupling device 124 may include apertures through which a fastening device such as a temporary anchorage device may be threaded to secure the dentition coupling device 124 to the patient's dentition. For example, the temporary anchorage devices may screw into the patient's bone tissue to secure the dentition coupling device 124.

In some embodiments, the dentition coupling device 124 includes one or more fiducial markers, such as hemispherical inserts, that can be used to establish a static relationship between the position of the clutch 120 and the patient's dentition. For example, the dentition coupling device 124 may include three fiducial markers disposed along its surface. The location of these fiducial markers can then be determined relative to the patient's dentition such as by capturing a physical impression of the patient with the clutch attached or using imaging techniques such as capturing a digital impression (e.g., with an intraoral scanner) or other types of images of the dentition and fiducial markers. Some embodiments of the dentition coupling device 124 do not include fiducial markers. One or more images or a digital impression of the patient's dentition captured while the dentition coupling device 124 is mounted may be aligned to one or more images or a digital impression of the patient's dentition captured while the dentition coupling device 124 is not mounted.

The position indicator system 128 is a system that is configured to be used to determine the position and orientation of the clutch 120. In some embodiments, the position indicator system 128 includes multiple fiducial markers. In some examples, the fiducial markers are spheres. Spheres work well as fiducial markers because the location of the center of the sphere can be determined in an image regardless of the angle from which the image containing the sphere was captured. The multiple fiducial markers may be disposed (e.g., non-collinearly) so that by determining the locations of each (or at least three) of the fiducial markers, the position and orientation of the clutch 120 can be determined. For example, these fiducial markers may be used to determine the position of the position indicator system 128 relative to the dentition coupling device 124, through which the position of the position indicator system 128 relative to the patient's dentition can be determined.

Some implementations of the position indicator system 128 do not include separate fiducial markers. In at least some of these implementations, structural aspects of the position indicator system 128 may be used to determine the position and orientation of the position indicator system 128. For example, one or more flat surfaces, edges, or corners of the position indicator system 128 may be imaged to determine the position and orientation of the position indicator system 128. In some implementations, an intraoral scanner is used to capture a three-dimensional model (or image) that includes a corner of the position indicator system 128 and at least part of the patient's dentition while the dentition coupling device 124 is mounted. This three-dimensional model can then be used to determine a relationship between the position indicator system 128 and the patient's dentition. The determined relationship may be a static relationship that defines the position and orientation of the position indicator system 128 relative to a three-dimensional model of the patient's dentition (e.g., based on the corner of the position indicator system 128 that was captured by the intraoral scanner).

In some embodiments, the position indicator system 128 includes a light source assembly that emits beams of light. The light source assembly may emit substantially collimated light beams (e.g., laser beams). In some embodiments, the light source assembly is rigidly coupled to the dentition coupling device 124 so that as the dentition coupling device 124 moves with the patient's dentition, the beams of light move. The position of the dentition coupling device 124 is then determined by capturing images of where the light beams intersect with various surfaces (e.g., translucent screens disposed around the patient). Embodiments that include a light source assembly are illustrated and described throughout.

The reference structure 122 is a structure that is configured to be worn by the patient so as to provide a point of reference to measure the motion of the clutch 120. In embodiments where the clutch 120 is configured to couple to the patient's lower dentition, the reference structure 122 is configured to mount elsewhere on the patient's head so that the motion of the clutch 120 (and the patient's mandible) can be measured relative to the rest of the patient's head. For example, the reference structure 122 may be worn on the upper dentition. Beneficially, when the reference structure 122 is mounted securely to the patient's upper dentition, the position of the reference structure 122 will not be impacted by the movement of the mandible (e.g., muscle and skin movement associated with the mandibular motion will not affect the position of the reference structure 122). Alternatively, the reference structure 122 may be configured to be worn elsewhere on the patient's face or head.

In some embodiments, the reference structure 122 is similar to the clutch 120 but configured to be worn on the dental arch opposite the clutch (e.g., the upper dentition if the clutch 120 is for the lower dentition). For example, the reference structure 122 shown in FIG. 2 includes a dentition coupling device 130 that may be similar to the dentition coupling device 124, and a position indicator system 134 that may be similar to the position indicator system 128.

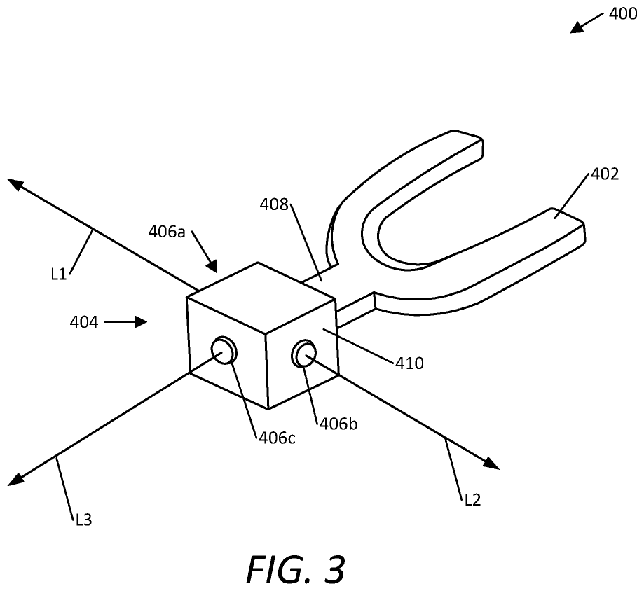

FIG. 3 illustrates an embodiment of a clutch 400. The clutch 400 is an example of the clutch 120. In this example, the clutch 400 includes a dentition coupling device 402 and a light source assembly 404, and an extension member 408. The dentition coupling device 402 is an example of the dentition coupling device 124, and the light source assembly 404 is an example of the position indicator system 128.

The light source assembly 404, which may also be referred to as a projector, is a device that emits light beams comprising light that is substantially collimated. Collimated light travels in one direction. A laser beam is an example of collimated light. In some embodiments, the light source assembly 404 includes one or more lasers. Although alternatives are possible, the one or more lasers may be semiconductor lasers such as laser diodes or solid-state lasers such as diode-pumped solid-state lasers.

In some embodiments, the light source assembly 404 comprises a first, second, and third light emitter. The first and second light emitters may emit substantially collimated light in parallel but opposite directions (i.e., the first and second light emitters may emit light in antiparallel directions) such as to the left and right of the patient when the clutch 400 is coupled to the patient's dentition. In some embodiments, the first and second light emitters are collinear or are substantially collinear (e.g., offset by a small amount such as less than 5 micrometers, less than 10 micrometers, less than 25 micrometers, less than 50 micrometers, or less than 100 micrometers). The third light emitter may emit substantially collimated light in a direction of a line that intersects with or substantially intersects with lines corresponding to the direction of the first and second light emitters. Lines that intersect share a common point. Lines that substantially intersect do not necessarily share a common point, but would intersect if offset by a small amount such as less than 5 micrometers, less than 10 micrometers, less than 25 micrometers, less than 50 micrometers, or less than 100 micrometers. In some embodiments, the third light emitter emits light in a direction that is perpendicular to the first and second light emitters, such as toward the direction the patient is facing.

In some embodiments, the third light emitter emits light in a direction that is offset from the direction of the first light emitter so as to be directed toward the same side of the patient as the direction of the first light emitter. For example, the third light emitter may be offset from the first light emitter by an offset angle of less than 90 degrees such that the light emitted by both the first light emitter and the second light emitter intersect with the same screen (e.g., a planar screen having a rectangular shape and being disposed on a side of the patient). The third light emitter may be offset from the first light emitter by an offset angle of between approximately 1 degree to 45 degrees. In some implementations, the offset angle is between 3 degrees and 30 degrees. In some implementations, the offset angle is between 5 degrees and 15 degrees. For example, the offset angle may be less than 10 degrees.

In some embodiments, one or more compensation factors are determined to compensate for an offset from the first and second light emitters being collinear, or an offset from the third light emitter emitting light in a direction of a line that intersects with the directions of the first and second light sources. A compensation factor may also be determined for the offset angle of the third light emitter with respect to the first and second light emitters. For example, an offset angle compensation factor may specify the angle between the direction of the third light emitter and a line defined by the first light emitter. In implementations in which the orientation of the third light emitter is directed perpendicular to or substantially perpendicular to the direction of the first light emitter, the offset angle compensation factor may be 90 degrees or approximately 90 degrees. In implementations in which the orientation of the third light emitter is directed toward a side of the patient, the offset angle compensation factor may, for example, be between approximately 5 and 45 degrees. The compensation factors may be determined specifically for each position indicator system manufactured to account for minor variation in manufacturing and assembly. The compensation factors may be stored in a datastore (such as on the motion determining device 106 or on a computer readable medium accessible by the motion determining device 106). Each position indicator system may be associated with a unique identifier that can be used to retrieve the associated compensation factor. The position indicator system 134 may include a label with the unique identifier or a barcode, QR code, etc. that specifies the unique identifier.

Some embodiments of the light source assembly 404 include a single light source and use one or more beam splitters such as prisms or reflectors such as mirrors to cause that light source to function as multiple light emitters by splitting the light emitted by that light source into multiple beams. In at least some embodiments, the emitted light emanates from a common point. As another example, some embodiments of the light source assembly 404 may comprise apertures or tubes through which light from a common source is directed. Some embodiments may include separate light sources for each of the light emitters.

In the example of FIG. 3, the light source assembly 404 includes light emitters 406a, 406b, and 406c (referred to collectively as light emitters 406) and a housing 410. The light emitter 406a is emitting a light beam L1, the light emitter 406b is emitting a light beam L2, and the light emitter 406c is emitting a light beam L3. The light beams L1 and L2 are parallel to each other, but directed in opposite directions. The light beam L3 is perpendicular to the light beams L1 and L2. In at least some embodiments, the housing 410 is configured to position the light emitters 406 so that the light beams L1, L2, and L3 are approximately co-planar with the occlusal plane of the patient's dentition. Although the light beam L3 is perpendicular to the light beams L1 and L2 in the example of FIG. 3, alternatives are illustrated and described with respect to at least FIGS. 22-28.

The housing 410 may be approximately cube shaped and includes apertures through which the light emitters 406 extend. In other embodiments, the light emitters do not extend through apertures in the housing 410 and instead light emitted by the light emitters 406 passes through apertures in the housing 410.

In the example of FIG. 3, the dentition coupling device 402 is rigidly coupled to the light source assembly 404 by an extension member 408. The extension member 408 extends from the dentition coupling device 402 and is configured to extend out of the patient's mouth when the dentition coupling device 402 is worn on the patient's dentition. In some embodiments, the extension member 408 is configured so as to be permanently attached to the light source assembly 404 such as by being formed integrally with the housing 410 or joined via welding or a permanent adhesive. In other embodiments, the extension member 408 is configured to removably attach to the light source assembly 404. Because the light source assembly 404 is rigidly coupled to the dentition coupling device 402, the position and orientation of the dentition coupling device 402 can be determined from the position and orientation of the light source assembly 404.

In some embodiments, the housing 410 and the dentition coupling device 402 are integral (e.g., are formed from a single material or are coupled together in a manner that is not configured to be separated by a user). In some embodiments, the housing 410 includes a coupling structure configured to removably couple to the extension member 408 of the dentition coupling device 402. In this manner, the dentition coupling device 402 can be a disposable component that may be custom fabricated for each patient, while the light source assembly 404 may be reused with multiple dentition coupling devices. In some embodiments, the housing 410 includes a connector that is configured to mate with a connector on the dentition coupling device 402. Additionally, the housing 410 may couple to the dentition coupling device 402 with a magnetic clasp. Some embodiments include a registration structure that is configured to cause the housing 410 to join with the dentition coupling device 402 in a repeatable arrangement and orientation. In some embodiments, the registration structure comprises a plurality of pins and corresponding receivers. In an example, the registration structure includes a plurality of pins disposed on the housing 410 and corresponding receivers (e.g., holes) in the dentition coupling device 402 (or vice versa). In some embodiments, the registration structure comprises a plurality of spherical attachments and a plurality of grooves. In one example, the registration structure includes three or more spherical attachments disposed on the housing 410 and two or more v-shaped grooves disposed on the dentition coupling device 402 that are disposed such that the spherical attachments will only fit into the grooves when the housing 410 is in a specific orientation and position relative to the dentition coupling device 402. In some implementations, the registration structure includes a spring-mounted pin or screw that serves as a detent to impede movement of the housing 410 with respect to the dentition coupling device 402.

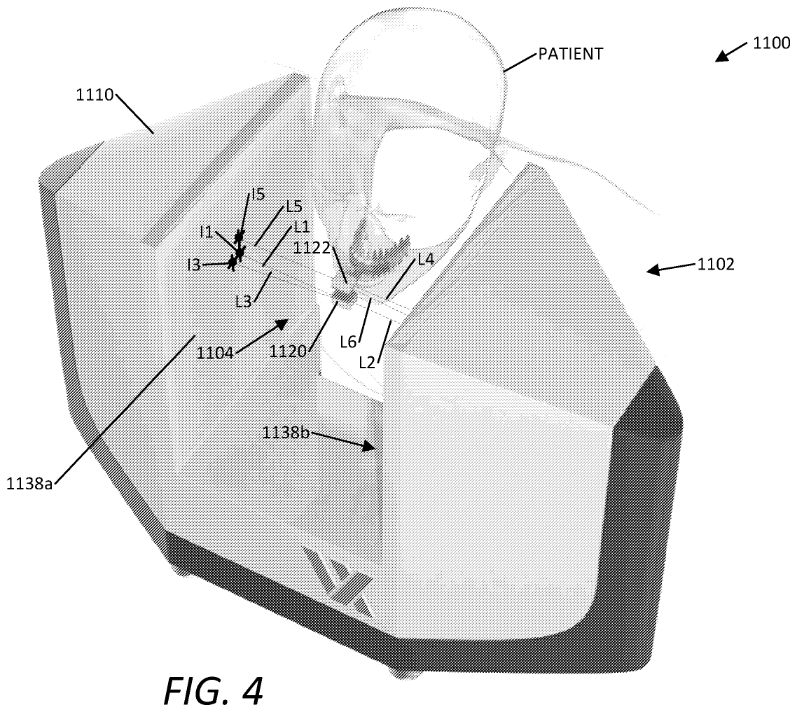

FIG. 4 illustrates an implementation of a motion capture system 1100 for capturing jaw movement in which only two screens are used. The motion capture system 1100 is an example of the system 100. The motion capture system 1100 includes an imaging system 1102 and a patient assembly 1104. In this example, the imaging system 1102 includes a housing 1110. The imaging system also includes screen 1138a and a screen 1138b (collectively referred to as screens 1138), which are positioned so as to be on opposite sides of the patient's face (e.g., screen 1138b to the left of the patient's face and screen 1138a to the right of the patient's face). In some implementations, a screen framework is disposed within the housing 1110 to position the screens 1138 with respect to each other and the housing 1110.

As can be seen in FIG. 4, this implementation does not include a screen disposed in front of the patient's face. Beneficially, by not having a screen in front of a patient's face, the system 1100 which may allow better access to the patient's face by a caregiver. Also shown, is patient assembly 1104 of the motion capture system 1100.

In at least some implementations, the patient assembly 1104 includes a clutch 1120 and a reference structure 1122, each of which include a light source assembly having three light emitters. The clutch 1120 is an example of the clutch 120 and the reference structure 1122 is an example of the reference structure 122. In FIG. 4, the clutch 1120 is attached to the patient's mandible (i.e., lower dentition) and is emitting light beams L1, L2, and L3. Light beams L1 and L3 are directed toward the screen 1138a, intersecting at intersection points I1 and I3, respectively. Light beam L2 is directed toward the screen 1138b. Although alternatives are possible, in this example, the light beams L1 and L3 are offset from each other by approximately 15 degrees. The light beams L1 and L2 are collinear and directed in opposite directions (i.e., L2 is offset from L1 by 180 degrees).

The reference structure 1122 is attached to the patient's maxilla (i.e., upper dentition) and is emitting light beams L4, L5, and L6. Light beams L4 and L6 are directed toward the screen 1138b. Light beam L5 is directed toward the screen 1138a, intersecting at intersection point I5. Although alternatives are possible, in this example, the light beams L4 and L6 are offset from each other by approximately 15 degrees. The light beams L4 and L5 are collinear and directed in opposite directions (i.e., L4 is offset from L5 by 180 degrees).

As the patient's dentition moves around, the clutch 1120 and the reference structure 1122 will move in concert with the patient's dentition, causing the lights beams to move and the intersection points to change. An optical sensing assembly of the motion capture system 1100 (e.g., cameras embedded within the housing 1110 of the system 1100 behind the screens 1138a and 1138b) may capture images of the screens 1138 so that the intersection points can be determined. The location of a first axis associated with the clutch 1120 may be identified based on the intersection points from the light beams L1 and L2. An intersection coordinate between the light beams L1 and L3 may then be determined based on the distance between the intersection points I1 and I3 on the screen 1138a. For example, the distance from the intersection point I1 along the first axis can be determined based on the distance between the points I1 and I3 and the angle between I1 and I3. As described in more detail elsewhere herein, the angle between I1 and I3 is determined for the clutch 1120 and may be stored in a data store, for example, on a non-transitory computer-readable storage medium. Using this distance, the intersection coordinate can be found, which will have a known relationship to the clutch 1120 and therefore the patient's dentition. A coordinate system for the clutch 1120 can be determined based on the intersection points too (e.g., a second axis is defined by the cross product of the first axis and a vector between the intersection points I1 and I3, and a third axis is defined by the cross product of the first axis and the second axis). In a similar manner, the position and orientation of the reference structure 1122 can be determined based on the intersection points of the light beams L4, L5, and L6 with the screens 1138a and 1138b.

In some implementations, three-dimensional coordinate systems for the clutch and the reference structure are determined using only two screens. In some implementations, the motion capture system includes only two screens and the motion capture system does not include a third screen. In some implementations, the imaging system captures images of only two screens. Some implementations identify intersection points using images captured of only two screens. For example, two intersection points from light beams emitted by a reference structure are identified on an image of the same screen.

In some implementations, a light emitter being oriented to emit light in a first direction toward the screen means the light emitter is oriented to emit light in a first direction toward the screen when the light emitter is attached to a patient (or other structure) and positioned for motion tracking with respect to the imaging system.

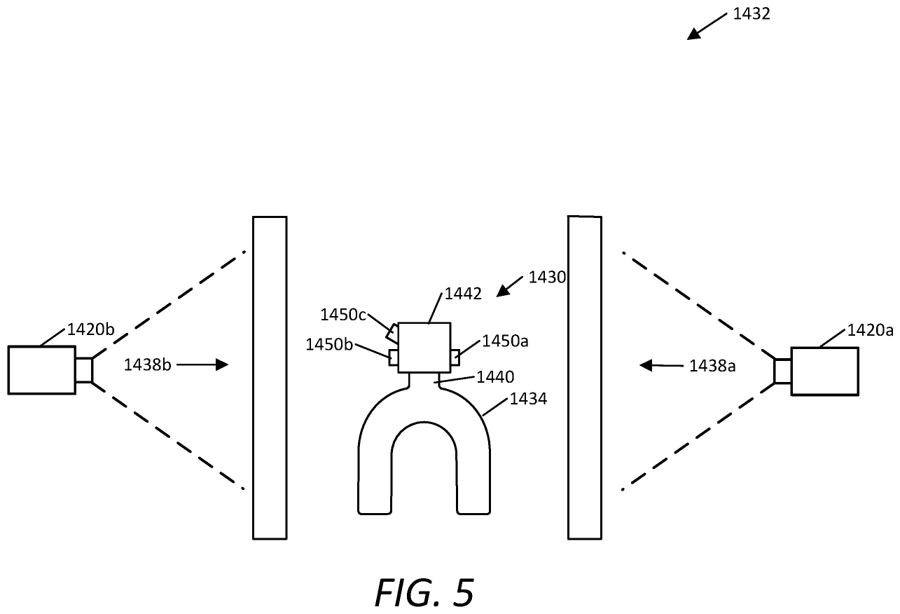

FIG. 5 illustrates a top view of an embodiment of a reference structure 1430 and an embodiment of an imaging system 1432. The reference structure 1430 is an example of the reference structure 1122. The imaging system 1432 is an example of the imaging system 1102.

The reference structure 1430 includes a dentition coupling device 1434, an extension member 1440, and a light source assembly 1442. The dentition coupling device 1434 is an example of the dentition coupling device 130 and may be similar to the example dentition coupling devices previously described with respect to embodiments of the clutch. The light source assembly 1442 is an example of the position indicator system 134. In this example, the light source assembly 1442 includes light emitters 1450a, 1450b, and 1450c (collectively referred to as light emitters 1450).

The dentition coupling device 1434 is configured to removably couple to the dentition of the patient. The dentition coupling device 1434 is coupled to the opposite arch of the patient's dentition as a clutch (e.g., the dentition coupling device 1434 couples to the maxillary arch when the clutch is coupled to the mandibular arch). In some embodiments, the dentition coupling device 1434 is coupled to the extension member 1440 that is configured to extend out through the patient's mouth when the dentition coupling device 1434 is coupled to the patient's dentition. The extension member 1440 may be similar to the extension member 408.

The imaging system 1432 includes screens 1438a and 1438b (referred to collectively as screens 1438), and cameras 1420a and 1420b (referred to collectively as cameras 1420). In this example, the screen 1438a is oriented parallel to the screen 1438b. In some embodiments. The imaging system 1432 may also include a screen framework (not shown) that positions the screens 1438 with respect to each other. For example, the screen framework may extend beneath the reference structure 1430 and couple to the bottoms of the screens 1438. Together, the screens 1438 and the screen framework are an example of the screen assembly 112. The cameras 1420 are an example of the optical sensing assembly 110.

The screens 1438 may be formed from a translucent material so that the points where the light beams emitted by the light source assembly 1442 intersect with the screens 1438 may be viewed from outside of the screens 1438. Images that include these points of intersection may be recorded by the cameras 1420. The motion determining device 106 may then analyze these captured images to determine the points of intersection of the light beams with the screens 1438 to determine the location of the light source assembly 1442. The position of the light source assembly of a clutch (not shown) may be determined in a similar manner.

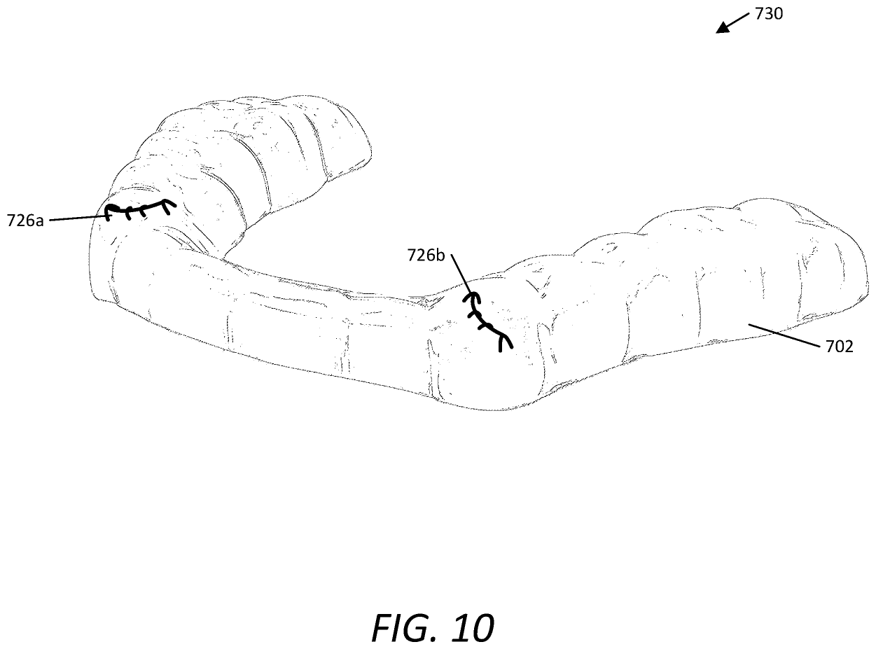

The cameras 1420 are positioned and oriented to capture images of the screens 1438. For example, the camera 1420a is positioned and oriented to capture images of the screen 1438a, and the camera 1420b is positioned and oriented to capture images of the screen 1438b. In some embodiments, the cameras 1420 are mounted to the screen framework so that they are position and orientation of the cameras 1420 are fixed with respect to the screens. For example, each of the cameras 1420 may be coupled to the screen framework by a camera mounting assembly such as is shown in FIG. 10. In this manner, the position and orientation of the cameras 1420 relative to the screens 1438 does not change if the screen framework is moved. In some implementations, the screen framework includes a housing (e.g., as shown at 1110 in FIG. 4), within which the cameras 1420 are disposed.

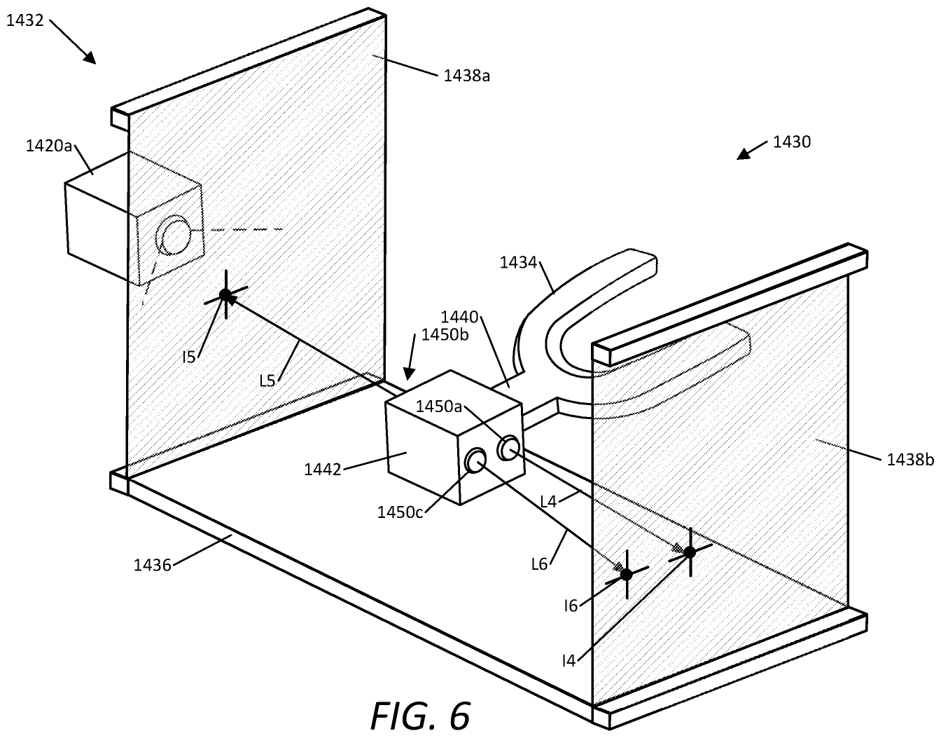

FIG. 6 illustrates a perspective view of the reference structure 1430 disposed between the screens 1438 of the imaging system 1432. The screens 1438 are joined together by a screen framework 1436 that positions and orients the screens 1438 with respect to one another. In this example, the light emitter 1450a is emitting a light beam L4, which intersects with the screen 1438b at intersection point I4; the light emitter 1450b is emitting a light beam L5, which intersects with the screen 1438a at intersection point I5; and the light emitter 1450c is emitting a light beam L6, which intersects with the screen 1438a at intersection point I6. As the position and orientation of the reference structure 1430 change relative to the screens 1438, the locations of at least some of the intersection points I4, I5, and I6 will change as well.

The camera 1420a captures images of the screen 1438a, including the intersection point I5 of the light beam L5 emitted by the light emitter 1450b. The camera 1420a may capture a video stream of these images. Similarly, although not shown in this illustration, the cameras 1420b captures images of the screens 1438b and the intersection points I4 and I6.

The captured images from the cameras 1420 are then transmitted to the motion determining device 106. The motion determining device 106 may determine the location of the intersection points I4, I5, and I6, and from those points the location of the light source assembly 1442. In some embodiments, a point of common intersection for the light beams L4, L5, and L6 is determined based on the location of the intersection points I4, I5, and I6 (e.g., the point at which the light beams intersect or would intersect if extended). Based on the determined locations of the light beams, the location and orientation of the reference structure 1430 relative to the screens 1438 can be determined.

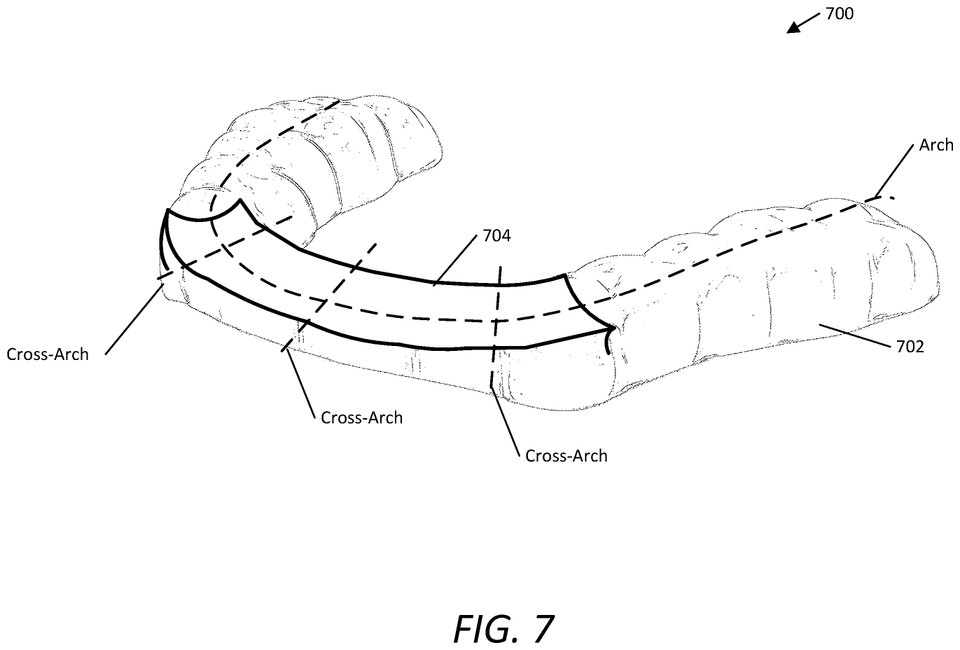

FIG. 7 is a schematic diagram of a motion-based dental splint 700 that includes a thin-shell aligner 702 and a contact surface 704. The thin-shell aligner 702 may, for example, have an interior surface and exterior surface. The interior surface may follow the contours of at least a portion of the patient's dentition. At least a portion of the exterior surface may also follow (or be based on) the contours of the patient's dentition. For example, the thin-shell aligner 702 may have a thickness of approximately 0.1-1 millimeters separating the interior surface from the exterior surface. In some implementations, the thin-shell aligner has a thickness of 0.5 millimeters. The exterior surface and the interior surface may be joined. For example, the exterior surface and the interior surface may be joined by an edge surface.

The interior surface may be an offset surface formed by offsetting the contours of the patient's dentition. The exterior surface may be an offset surface formed by offsetting the interior surface.

The contact surface 704 may be a part of the exterior surface of the thin-shell aligner 702. For example, the exterior surface may include the contact surface and a contour-following surface. The contour-following surface may represent the portion of the exterior surface other than the contact surface 704. The contour following surface of the exterior surface may be an offset surface formed by offsetting the interior surface. At the contact surface 704, the thin-shell aligner 702 may be thicker than in the contour-following region.

In this example, the contact surface 704 extends from cuspid to cuspid. The contact surface 704 may have a concave shape in the cross-arch dimension (direction). As used herein, the cross-arch dimension (direction) refers to a direction that is perpendicular or approximately perpendicular to the dental arch. For example, the cross-arch dimension (direction) corresponds approximately to the labial-lingual dimension of the anterior teeth and the buccal-lingual dimension of posterior teeth. An example of the arch and cross-arch dimension (direction) are shown in FIG. 7 for illustrative purposes.

This concave shape may be defined based on the motion-data. In some embodiments, the contact surface 704 may be raised in the incisal (anterior) region and may slope away toward the posterior.

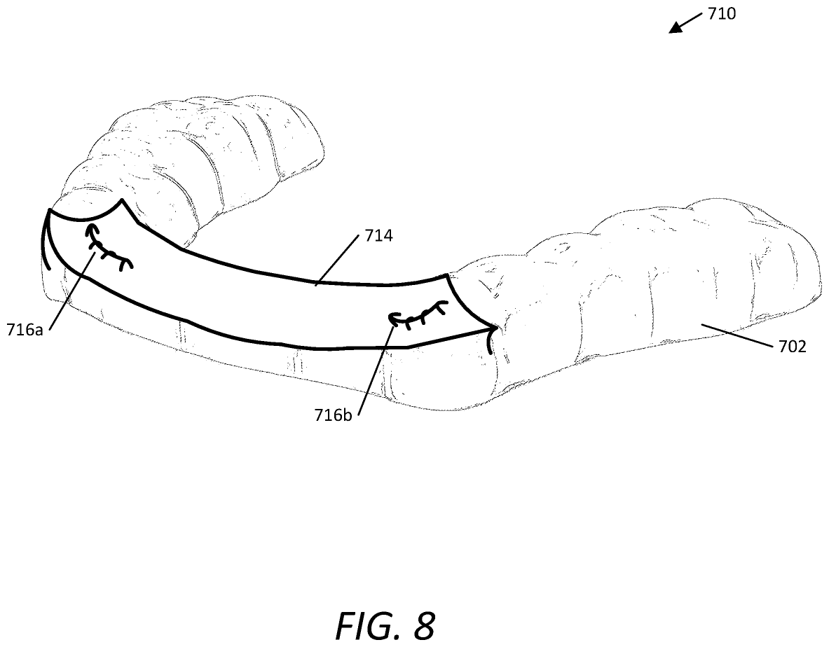

FIG. 8 is a schematic diagram of a motion-based dental splint 710 that includes the thin-shell aligner 702 and a contact surface 714. In this example, the contact surface 714 is similar to the contact surface 704 and extends from cuspid to cuspid. The contact surface 714 includes contact ridges 716a and 716b (referred to collectively as contact ridges 716). The contact ridges 716 may be formed by moving contact regions on the cuspids opposing the splint through an excursive movement based on motion data.

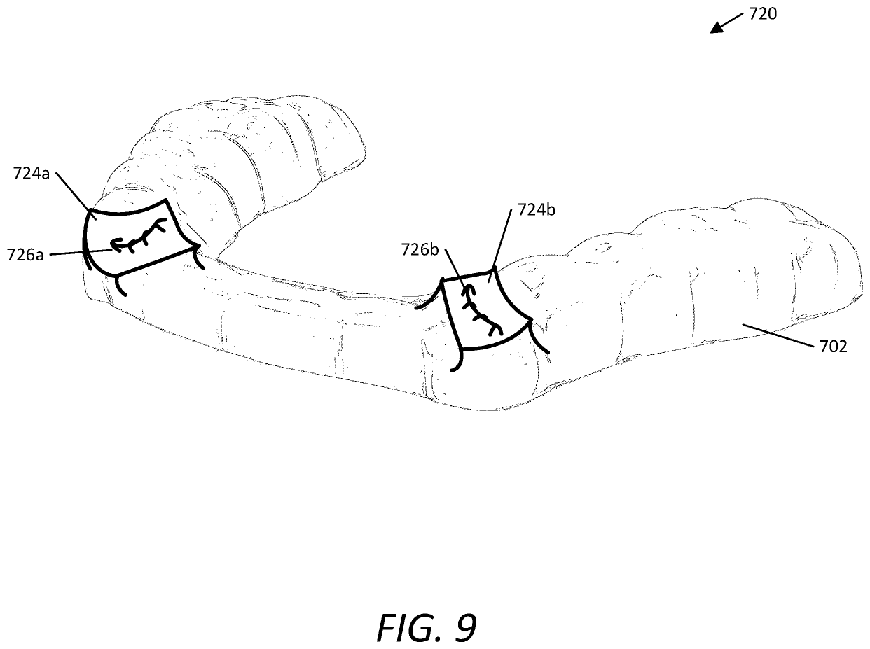

FIG. 9 is a schematic diagram of a motion-based dental splint 720 that includes the thin-shell aligner 702 and contact surfaces 724a and 724b (referred to collectively as contact surface 724). In this example, the contact surfaces 724a and 724b each cover a cuspid. The contact surface 724 may have a concave shape in the cross-arch dimension. The contact surface 724a includes a contact ridge 726a and the contact surface 724b includes a contact ridge 726b. The contact ridges 726a and 726b may be formed by moving contact regions on the cuspids opposing the splint through a protrusive movement based on motion data.

FIG. 10 is a schematic diagram of a motion-based dental splint 730 that includes the thin-shell aligner 702 and the contact ridges 726a and 726b. In this example, the contact ridges 726a and 726b are joined directly to the thin-shell aligner 702.

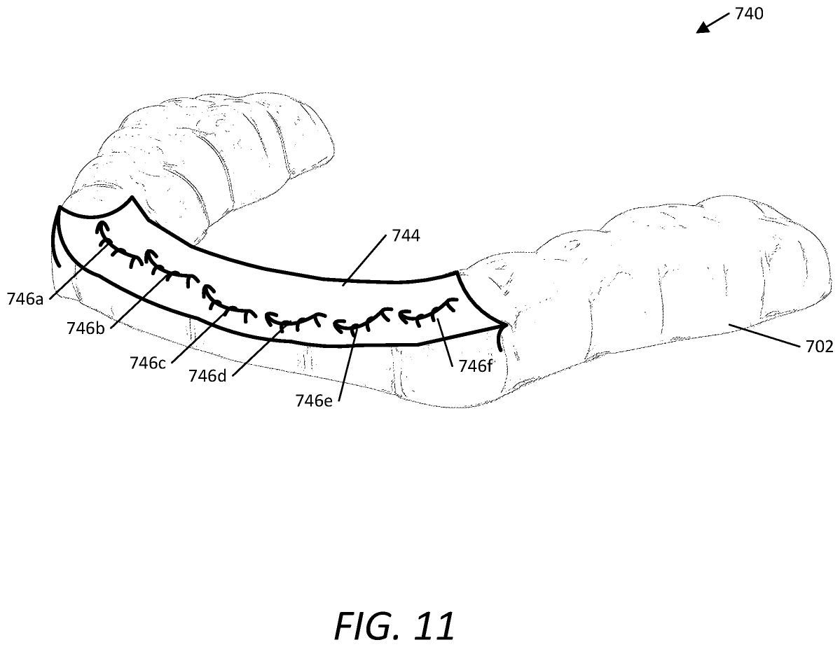

FIG. 11 is a schematic diagram of a motion-based dental splint 740 that includes the thin-shell aligner 702 and a contact surface 744. In this example, the contact surface 744 is similar to the contact surface 704 and extends from cuspid to cuspid. The contact surface 744 includes contact ridges 746a, 746b, 746c, 746d, 746e, and 746f (referred to collectively as contact ridges 746). The contact ridges 746 may be formed by moving contact regions on the anterior teeth opposing the splint through an excursive movement based on motion data.

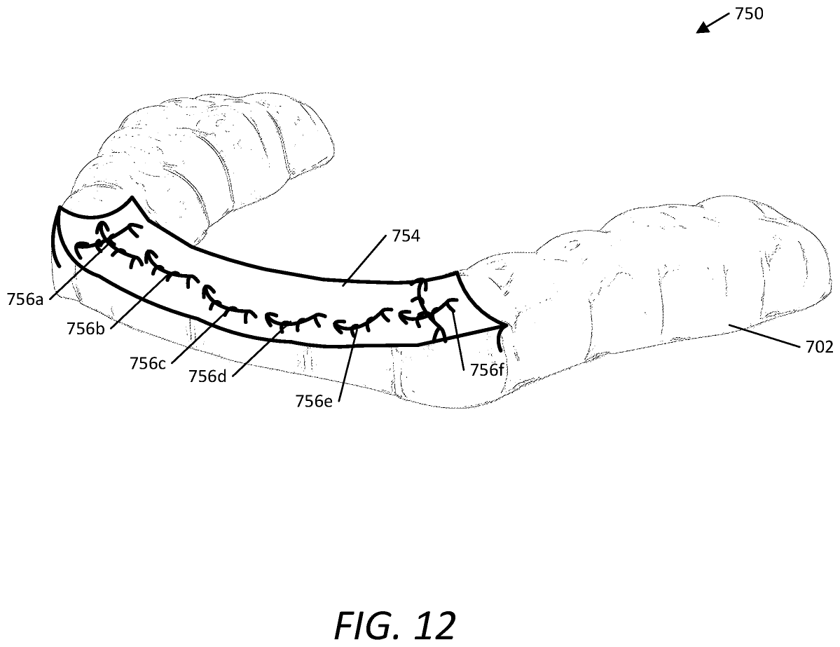

FIG. 12 is a schematic diagram of a motion-based dental splint 750 that includes the thin-shell aligner 702 and a contact surface 754. In this example, the contact surface 754 is similar to the contact surface 704 and extends from cuspid to cuspid. The contact surface 754 includes contact ridges 756a, 756b, 756c, 756d, 756e, and 756f (referred to collectively as contact ridges 756). The contact ridges 756 may be formed by moving contact regions on the anterior teeth opposing the splint through various jaw movements based on motion data. In this example, the contact ridges 756a and 756f are formed by moving contact regions on the cuspids opposing the splint through both excursive and protrusive movements based on motion data, and the contact ridges 756b, 756c, 756d, and 756e are formed by moving contact regions on the incisors opposing the splint through excursive movements based on motion data.

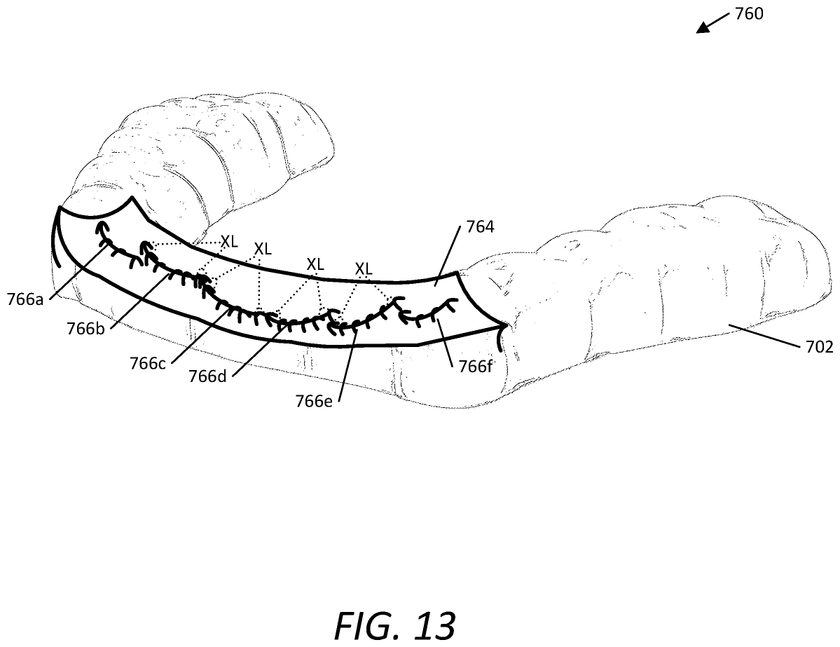

FIG. 13 is a schematic diagram of a motion-based dental splint 760 that includes the thin-shell aligner 702 and a contact surface 764. In this example, the contact surface 764 is similar to the contact surface 744 and extends from cuspid to cuspid. The contact surface 764 includes contact ridges 766a, 766b, 766c, 766d, 766e, and 766f (referred to collectively as contact ridges 766). The contact ridges 766 may be formed by moving contact regions on the anterior teeth opposing the splint through an excursive movement based on motion data. In this example, the contact ridges 766a and 766f may be similar to or identical to the contact ridges 746a and 746f, respectively, shown in FIG. 11. The contact ridges 766b, 766c, 766d, and 766e may be similar to the contact ridges 746b, 746c, 746d, and 746e, respectively, except that the contact ridges 766b, 766c, 766d, and 766e may be longer as they include extreme lateral regions that are labeled XL in this figure. The extreme lateral regions (which may also be referred to as cross-over regions) may extend on one or both sides of the contact ridges. The extreme lateral regions may be positioned to contact some or all of the patient's teeth when the patient's teeth move from a standard bite to a cross-bite (e.g., the lingual cusps of the patient's upper molars cross-over the buccal cusps of the corresponding lower teeth). Here, the extreme lateral regions are shown for the patient's anterior teeth but not for the patient's cuspids as the patient's cuspids may be out of contact during extreme lateral movement. In some embodiments, the contact surface 764 may include regions for the incisors to nest in during cross-over motions that are designed based on motion data.

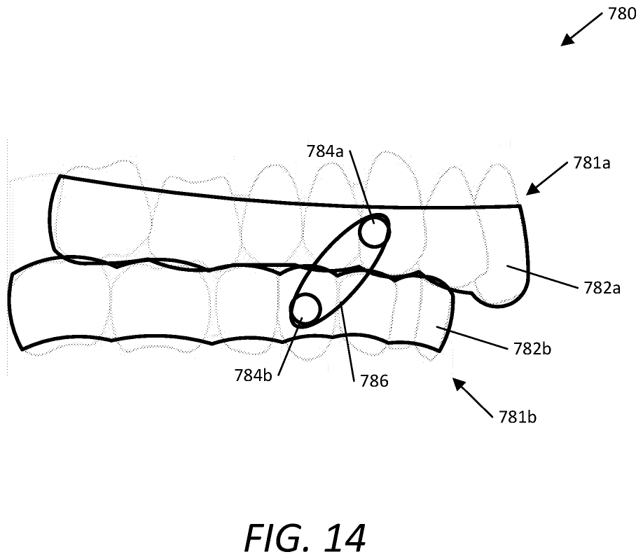

FIG. 14 is a schematic diagram of a side view of a dental splint arrangement 780 that includes a dental splints 781a and 781b and joining structure 786. The dental splint 780a includes a thin-shell aligner 782a and an attachment structure 784a. The dental splint 780b includes a thin-shell aligner 782b and an attachment structure 784b. Although not shown, at least one of the dental splints 780a and 780b may also include a contact surface similar to the contact surfaces illustrated and described with respect to at least one of FIGS. 7-13. The joining structure 786 joins the dental splints 781a and 781b. The joining structure 786 may provide a spring-like force to pull the dental splint 781b forward with respect to the dental splint 781a. Examples of the joining structure 786 include elastic bands and springs. The joining structure 786 may attach to the attachment structure 784a and the attachment structure 784b. Examples of the attachment structures 784a and 784b include ridges, buttons, and hooks upon which ends for the structure 786 can be secured.

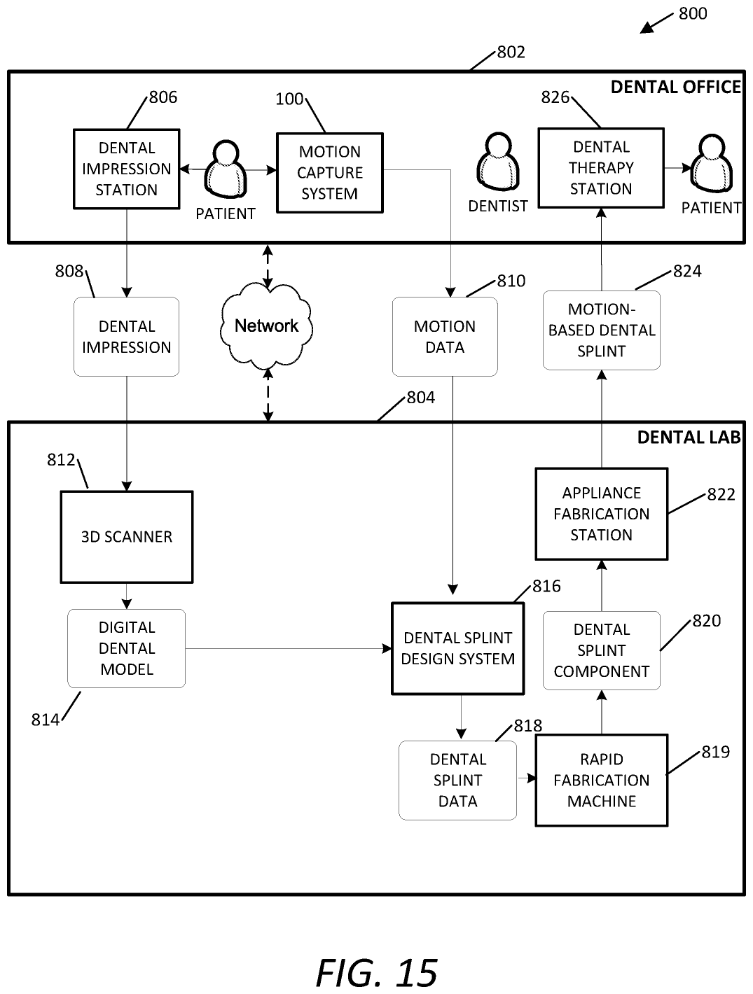

FIG. 15 is a schematic block diagram illustrating an example of a system 800 for using jaw motion captured by the motion capture system 100 to fabricate a motion-based dental splint 824 or provide dental therapy. In this example, the system 800 includes a dental office 802 and a dental lab 804.

The example dental office 802 includes a dental impression station 806, the motion capture system 100, and a dental therapy station 826. Although shown as a single dental office in this figure, in some embodiments, the dental office 802 includes multiple dental offices. For example, in some embodiments, one or both of the dental impression station 806 and the motion capture system 100 are in a different dental office than the dental therapy station 826. Further, in some embodiments, one or more of the dental impression station 806, the motion capture system 100, and the dental therapy station 826 are not in a dental office.

The example dental impression station 806 generates a dental impression 808 of the dentition of the patient. The dental impression 808 is a geometric representation of the dentition of the patient. In some embodiments, the dental impression 808 is a physical impression captured using an impression material, such as sodium alginate, or polyvinylsiloxane. In other embodiments, other impression materials are used as well. In some embodiments, the dental impression is captured by an impression device of the motion capture system 100. In other words, some embodiments do not include a dental impression station 806 that is separate from the motion capture system 100.

In some embodiments, the dental impression 808 is a digital impression. In some embodiments, the digital impression is represented by one or more of a point cloud, a polygonal mesh, a parametric model, or voxel data. In some embodiments, the digital impression is generated directly from the dentition of the patient, using for example an intraoral scanner. Example intraoral scanners include the TRIOS Intra Oral Digital Scanner, the Lava Chairside Oral Scanner C.O.S., the Cadent iTero, the Cerec AC, the Cyrtina IntraOral Scanner, and the Lythos Digital Impression System from Ormco. In other embodiments, a digital impression is captured using other imaging technologies, such as computed tomography (CT), including cone beam computed tomography (CBCT), ultrasound, and magnetic resonance imaging (MRI). In yet other embodiments, the digital impression is generated from a physical impression by scanning the impression or plaster model of the dentition of the patient created from the physical impression. Examples of technologies for scanning a physical impression or model include three-dimensional laser scanners and computed tomography (CT) scanners. In yet other embodiments, digital impressions are created using other technologies.

The motion capture system 100 has been described previously and captures a representation of the movement of the dental arches relative to each other. In some embodiments, the motion capture system 100 generates motion data 810 representing the movement of the arches relative to one another. In some embodiments, the motion capture system 100 generates the motion data 810 from optical measurements of the dental arches that are captured while the dentition of the patient is moved. In some embodiments, the optical measurements are extracted from images or video data recorded while the dentition of the patient is moved. Additionally, in some embodiments, the optical measurements are captured indirectly. For example, in some embodiments, the optical measurements are extracted from images or video data of one or more devices (e.g., the patient assembly 104) that are secured to a portion of the dentition of the patient. In other embodiments, the motion data 810 is generated using other processes. Further, in some embodiments, the motion data 810 includes transformation matrices that represent the position and orientation of the dental arches. The motion data 810 may include a series of transformation matrices that represent various motions or functional paths of movement for the patient's dentition. Other embodiments of the motion data 810 are possible as well.

In some embodiments, still images are captured of the patient's dentition while the dentition of the patient is positioned in a plurality of bite locations. In some embodiments, image processing techniques are used to determine the positions of the patient's upper and lower arches relative to each other (either directly or based on the positions of the attached patient assembly 104). In some embodiments, the motion data 810 is generated by interpolating between the positions of the upper and lower arches determined from at least some of the captured images.

The motion data 810 may be captured with the patient's jaw in various static positions or moving through various motions. For example, the motion data 810 may include a static measurement representing a centric occlusion (i.e., the patient's mandible closed with teeth fully engaged) or centric relation (i.e., the patient's mandible nearly closed, just before any shift occurs that is induced by tooth engagement or contact) bite of a patient. The motion data 810 may also include static measurements or sequences of data corresponding to protrusive (i.e., the patient's mandible being shifted forward while closed), lateral excursive (i.e., the patient's mandible shifted/rotated left and right while closed), hinging (i.e., the patient's mandible opening and closing without lateral movement), chewing (i.e., the patient's mandible chewing naturally to, for example, determine the most commonly used tooth contact points), and border movements (i.e., the patient's mandible is shifted in all directions while closed, for example, to determine the full range of motion) of the patient's jaw. This motion data 810 may be used to determine properties of the patient's temporomandibular joint (TMJ). For example, hinging motion of the motion data 810 may be used to determine the location of the hinge axis of the patient's TMJ.

The example dental lab 804 includes a 3D scanner 812, a design system 816, a rapid fabrication machine 819, and an appliance fabrication station 822. Although shown as a single dental lab in this figure, in some embodiments, the dental lab 804 comprises multiple dental labs. For example, in some embodiments, the 3D scanner 812 is in a different dental lab than one or more of the other components shown in the dental lab 804. Further, in some embodiments, one or more of the components shown in the dental lab 804 are not in a dental lab. For example, in some embodiments, one or more of the 3D scanner 812, dental splint design system 816, rapid fabrication machine 819, and appliance fabrication station 822 are in the dental office 802. Additionally, some embodiments of the system 800 do not include all of the components shown in the dental lab 804.

The example 3D scanner 812 is a device configured to create a three-dimensional digital representation of the dental impression 808. In some embodiments, the 3D scanner 812 generates a point cloud, a polygonal mesh, a parametric model, or voxel data representing the dental impression 808. In some embodiments, the 3D scanner 812 generates a digital dental model 814. In some embodiments, the 3D scanner 812 comprises a laser scanner, a touch probe, or an industrial CT scanner. Yet other embodiments of the 3D scanner 812 are possible as well. Further, some embodiments of the system 800 do not include the 3D scanner 812. For example, in some embodiments of the system 800 where the dental impression station 806 generates a digital dental impression, the 3D scanner 812 is not included.

The dental splint design system 816 is a system that is configured to generate the dental splint data 818. In some embodiments, the dental splint data 818 is three-dimensional digital data that represents the dental splint component 820 and is in a format suitable for fabrication using the rapid fabrication machine 819.

In some embodiments, the dental splint design system 816 comprises a computing device including user input devices. The design system 816 may include computer-aided-design (CAD) software that generates a graphical display of the dental splint data 818 and allows an operator to interact with and manipulate the dental splint data 818. For example, the dental splint design system 816 may include digital tools that mimic the tools used by a laboratory technician to physically design a dental appliance. For example, some embodiments include a tool to move the patient's dentition according to the motion data 810. Additionally, in some embodiments, the dental splint design system 816 includes a server that partially or fully automates the generation of designs of the dental splint data 818, which may use the motion data 810.

The dental splint design system 816 may be usable to design one or more dental appliance and/or dental treatment concurrently. The motion data 810 may be used to evaluate the interaction between the one or more dental appliance and/or dental treatment. This may be particularly beneficial in designing complex appliances and planning complex dental treatments such as implant supported denture systems.

In some embodiments, the rapid fabrication machine 819 comprises one or more three-dimensional printers, such as the ProJet line of printers from 3D Systems, Inc. of Rock Hill, S.C. Another example of the rapid fabrication machine 819 is stereolithography equipment. Yet another example of the rapid fabrication machine 819 is a milling device, such as a computer numerically controlled (CNC) milling device. In some embodiments, the rapid fabrication machine 819 is configured to receive files in the STL format. Other embodiments of the rapid fabrication machine 819 are possible as well.

Additionally, in some embodiments, the rapid fabrication machine 819 is configured to use the dental splint data 818 to fabricate the dental splint component 820. In some embodiments, the dental splint component 820 is a physical component that is configured to be used as part or all of the motion-based dental splint 824. For example, in some embodiments, the dental splint component is milled from a biocompatible plastic material or another material that is used directly as a dental splint. In other embodiments, the dental splint component 820 is a mold formed from wax or another material and is configured to be used indirectly (e.g., through a vacuum forming process) to fabricate the motion-based dental splint 824. Further, in some embodiments, the dental splint component 820 is formed using laser sintering technology.

In some embodiments, the appliance fabrication station 822 fabricates a motion-based dental splint 824 for the patient. In some embodiments, the appliance fabrication station 822 uses the dental splint component 820 produced by the rapid fabrication machine 819. In some embodiments, the motion-based dental splint 824 is a temporomandibular disorder (TMD) splint. Other embodiments of the motion-based dental splint 824 are possible as well. In some embodiments, the motion-based dental splint 824 is formed from an acrylic or plastic material. In some embodiments, the dental impression 808 is used in the fabrication of the motion-based dental splint 824. In some embodiments, the dental impression 808 is used to form a plaster model of the dentition of the patient. Additionally, in some embodiments, a model of the dentition of the patient is generated by the rapid fabrication machine 819. In some embodiments, the appliance fabrication station 822 includes equipment and processes to perform some or all of the techniques used in traditional dental laboratories to generate dental appliances. Other embodiments of the appliance fabrication station 822 are possible as well.

In some embodiments, the motion-based dental splint 824 is seated in the mouth of the patient in the dental therapy station 826 by a dentist. In some embodiments, the dentist confirms that the occlusal surface of the motion-based dental splint 824 is properly defined by instructing the patient to engage in various bites or perform various jaw motions.

Although many of the embodiments of the dental lab discussed herein receive motion data 810 from the motion capture system 100, alternatives are possible. The motion data 810 may be received from any system that captures motion data of the patient or an existing record of the patient's motion.

Additionally, in some embodiments, the dental office 802 is connected to the dental lab 804 via a network. In some embodiments, the network is an electronic communication network that facilitates communication between the dental office 802 and the dental lab 804. An electronic communication network is a set of computing devices and links between the computing devices. The computing devices in the network use the links to enable communication among the computing devices in the network. The network can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network includes various types of links. For example, the network can include one or both of wired and wireless links, including Bluetooth, ultra-wideband (UWB), 802.11, ZigBee, and other types of wireless links. Furthermore, in various embodiments, the network is implemented at various scales. For example, the network can be implemented as one or more local area networks (LANs), metropolitan area networks, subnets, wide area networks (such as the Internet), or can be implemented at another scale.

FIG. 16 is an example process 850 for designing a dental splint based on captured jaw motion. In some embodiments, the process 850 is performed by the system 800.

At operation 852, an impression of a patient's dentition is acquired. In some aspects, the impression is captured using a digital or physical impressioning technique. Alternatively, the impression is acquired from a storage location such as a database that stores dental impression data (e.g., from a previous patient visit).

At operation 854, jaw motion data for the patient is captured. The motion data may for example be captured using the motion capture system 100 or another system for capturing patient motion. Some or all of the motion data may also be inferred based on an actual or inferred location of the patient's condyle with respect to the position of the patient's dentition in the impression received at operation 852. Alternatively, the motion data is acquired from a storage location such as a database that stores dental motion data (e.g., from a previous patient visit).

At operation 856, a thin-shell appliance is generated for the patient based on the impression. In some embodiments, the thin-shell appliance is formed for one arch of the patient's dentition (referred to herein as the splint arch). The thin-shell appliance may be generated by identifying a region of a surface of the splint arch, offsetting that region by a first amount to generate an inner surface of the thin-shell appliance, offsetting that region by a second amount to generate an outer surface of the thin-shell appliance, and joining the inner surface and the outer surface. For example, the region may be identified based on a desired tooth coverage for the thin-shell appliance, such as coverage of all of the teeth or coverage of just the anterior teeth. The region may further be identified to include the surfaces of the teeth starting from the gingival margin, the heights of contour, or a location in-between. The region may be identified automatically, based on user input, or any combination thereof. Offsetting the surface may include generating a copy of the surface of the identified region of the splint arch and moving each vertex of the copied surface out by a specified amount in a direction normal to the surface. The offsetting may also include various operations to ensure the surface remains manifold (i.e., does not intersect with itself) in regions where the surface normal direction changes rapidly. Non-uniform offsets may be applied in some implementations.

At operation 858, a contact surface is generated based on the motion data. The contact surface may be a single continuous surface or multiple disjoint surfaces. In some implementations, the contact surface is generated based on moving the arch of the patient's dentition opposing the splint arch (referred to herein as the opposing arch) based on the motion data. The opposing arch may be moved relative to the splint arch. In some implementations, one or more contact regions are identified on the opposing arch. The contact regions may include, for example, regions of the opposing arch that should be in contact with the splint during a specific bite motion when the patient is wearing the splint. The contact regions may include portions of the occlusal surfaces (such as cusp tips or incisal edges) of the patient's teeth on the opposing arch. The contact regions may be identified automatically or based on user input. In some implementations, a user interface is provided to allow a dentist or other care provider to specify which teeth should be in contact.

In some implementations, vertices associated with contact regions may be identified. The contact surface may be generated based on a path swept by those vertices as the opposing arch is moved through a specific motion path relative to the splint arch.

In some implementations, the opposing arch is opened by a specified amount before moving through specific motion path to generate contact surface. For example, the opposing arch may be opened from a record closed bite (i.e., centric occlusion) position by performing a hinge motion to bring the opposing arch out of contact by the specified amount. In some implementations, the opposing arch may be initially positioned in a captured open bite (i.e., centric relation).

At operation 860, the contact surface is joined with the thin-shell appliance. Various techniques may be used to join the contact surface to the thin-shell appliance. For example, the contact surface may be extended from a surface to a solid mesh (e.g., by joining the contact surface to a copy of the contact surface that is repositioned toward the gingival). The solid mesh may then be joined to the thin-shell appliance with a Boolean operation (e.g., a union). The contact surface may also be joined to the thin-shell appliance by deforming the outer surface of the thin-shell appliance to meet the contact surface.

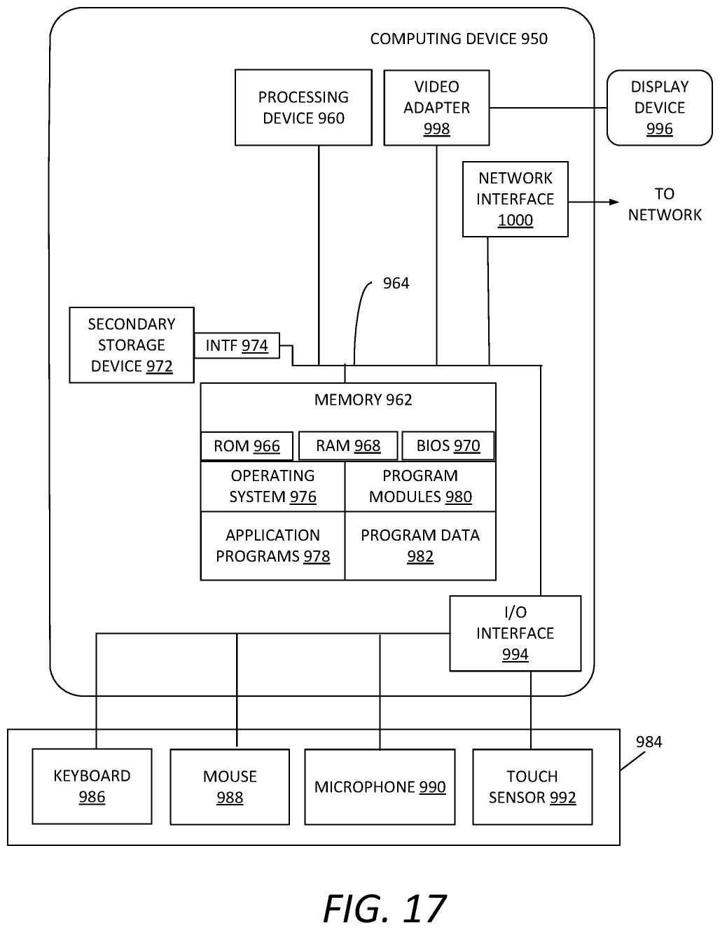

FIG. 17 illustrates an example architecture of a computing device 950 that can be used to implement aspects of the present disclosure, including any of the plurality of computing devices described herein, such as a computing device of the motion determining device 106, the design system 816, or any other computing devices that may be utilized in the various possible embodiments.

The computing device illustrated in FIG. 17 can be used to execute the operating system, application programs, and software modules described herein.

The computing device 950 includes, in some embodiments, at least one processing device 960, such as a central processing unit (CPU). A variety of processing devices are available from a variety of manufacturers, for example, Intel or Advanced Micro Devices. In this example, the computing device 950 also includes a system memory 962, and a system bus 964 that couples various system components including the system memory 962 to the processing device 960. The system bus 964 is one of any number of types of bus structures including a memory bus, or memory controller; a peripheral bus; and a local bus using any of a variety of bus architectures.

Examples of computing devices suitable for the computing device 950 include a desktop computer, a laptop computer, a tablet computer, a mobile computing device (such as a smart phone, an iPod® or iPad® mobile digital device, or other mobile devices), or other devices configured to process digital instructions.

The system memory 962 includes read only memory 966 and random-access memory 968. A basic input/output system 970 containing the basic routines that act to transfer information within computing device 950, such as during start up, is typically stored in the read only memory 966.

The computing device 950 also includes a secondary storage device 972 in some embodiments, such as a hard disk drive, for storing digital data. The secondary storage device 972 is connected to the system bus 964 by a secondary storage interface 974. The secondary storage devices 972 and their associated computer readable media provide nonvolatile storage of computer readable instructions (including application programs and program modules), data structures, and other data for the computing device 950.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory computer-readable media. Additionally, such computer readable storage media can include local storage or cloud-based storage.

A number of program modules can be stored in secondary storage device 972 or system memory 962, including an operating system 976, one or more application programs 978, other program modules 980 (such as the software engines described herein), and program data 982. The computing device 950 can utilize any suitable operating system, such as Microsoft Windows™, Google Chrome™ OS or Android, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 950 through one or more input devices 984. Examples of input devices 984 include a keyboard 986, mouse 988, microphone 990, and touch sensor 992 (such as a touchpad or touch sensitive display). Other embodiments include other input devices 984. The input devices are often connected to the processing device 960 through an input/output interface 994 that is coupled to the system bus 964. These input devices 984 can be connected by any number of input/output interfaces, such as a parallel port, serial port, game port, or a universal serial bus. Wireless communication between input devices and the interface 994 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 996, such as a monitor, liquid crystal display device, projector, or touch sensitive display device, is also connected to the system bus 964 via an interface, such as a video adapter 998. In addition to the display device 996, the computing device 950 can include various other peripheral devices (not shown), such as speakers or a printer.

When used in a local area networking environment or a wide area networking environment (such as the Internet), the computing device 950 is typically connected to the network through a network interface 1000, such as an Ethernet interface or WiFi interface. Other possible embodiments use other communication devices. For example, some embodiments of the computing device 950 include a modem for communicating across the network.

The computing device 950 typically includes at least some form of computer readable media. Computer readable media includes any available media that can be accessed by the computing device 950. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 950.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 17 is also an example of programmable electronics, which may include one or more such computing devices, and when multiple computing devices are included, such computing devices can be coupled together with a suitable data communication network so as to collectively perform the various functions, methods, or operations disclosed herein.

Some non-limiting examples are provided below:

Example 1: A dental appliance for a dental arch of a patient, the appliance comprising: an interior surface shaped to fit to contours of at least one tooth of the dental arch; and an exterior surface that includes at least one contact surface formed based on motion data.

Example 2: The dental appliance of example 1, wherein the exterior surface includes a contour-following surface that follows the contours of at least one tooth of the dental arch.

Example 3: The dental appliance of example 2, wherein the contour-following surface is an offset surface of the interior surface.

Example 4: The dental appliance of any one of examples 1-3, wherein the contact surface is disposed in an anterior region of the dental arch.

Example 5: The dental appliance of example 4, wherein the contact surface extends from cuspid to cuspid.

Example 6: The dental appliance of any one of examples 2-5, wherein the contour-following surface is disposed in at least one posterior region of the dental arch.

Example 7: The dental appliance of any one of examples 1-6, wherein the contact surface has a concave shape in a cross-arch dimension.

Example 8: The dental appliance of any one of examples 1-7, wherein the contact surface has shape that corresponds to a shape formed by sweeping a point from an opposing dental arch through a motion path relative to the dental arch, the motion path being based on the motion data.

Example 9: The dental appliance of example 8, wherein the contact surface has shape that corresponds to a shape formed by sweeping a point from the opposing dental arch through a protrusive motion path relative to the dental arch.

Example 10: The dental appliance of any of examples 8 or 9, wherein the contact surface has shape that corresponds to a shape formed by hinging the opposing arch open by a specific amount and sweeping a point from the opposing dental arch through a motion path relative to the dental arch.

Example 11: The dental appliance of any one of examples 1-10, wherein the contact surface is offset in an occlusal direction by a predetermined amount.

Example 12: The dental appliance of example 11, wherein the predetermined amount is based on a centric relation bite of the patient.

Example 13: The dental appliance of any one of examples 1-12, wherein the contact surface is raised occlusally in an anterior region and slopes away toward a posterior region.

Example 14: The dental appliance of any one of examples 1-13, wherein the contact surface is shaped so that when the dental appliance is worn by the patient, some or all of the opposing dental arch remains in contact with the contact surface throughout a motion path from the motion data.

Example 15: The dental appliance of example 14, wherein the contact surface is shaped so that when the dental appliance is worn by the patient, at least one cuspid of an opposing dental arch of the patient remains in contact with the contact surface throughout a protrusive motion path from the motion data.

Example 16: The dental appliance of example 14, wherein the contact surface is shaped so that when the dental appliance is worn by the patient, at least one anterior tooth of an opposing dental arch of the patient remains in contact with the contact surface throughout an excursive motion path from the motion data.

Example 17: The dental appliance of example 16, wherein the at least one anterior tooth includes an incisor.

Example 18: The dental appliance of any one of examples 1-17, wherein the contact surface is shaped so that when the dental appliance is worn by the patient, contact force is balanced approximately evenly across all opposing teeth that are in contact.

Example 19: The dental appliance of any one of examples 1-18, wherein the contact surface includes at least one contact ridge.

Example 20: The dental appliance of example 19, wherein the at least one contact ridge includes a raised ridge on the contact surface.

Example 21: The dental appliance of example 20, wherein the at least one contact ridge includes a cuspid contact ridge that is positioned based on a contact region of a cuspid of an opposing dental arch of the patient.

Example 22: The dental appliance of example 21, wherein the cuspid contact ridge has a shape that corresponds to a shape formed by sweeping the contact region through a protrusive motion path relative to the dental arch.

Example 23: The dental appliance of any one of examples 21 or 22, wherein the at least one contact ridge includes an incisor contact ridge that is positioned based on a contact region of an incisor of an opposing dental arch of the patient.

Example 24: The dental appliance of example 23, wherein the incisor contact ridge has a shape that corresponds to a shape formed by sweeping the contact region through an excursive motion path relative to the dental arch.

Example 25: The dental appliance of any one of examples 23 or 24, wherein the incisor contact ridge includes an extreme lateral region.

Example 26: The dental appliance of example 25, wherein the extreme lateral region has a shape that corresponds to a shape formed by sweeping the contact region from a standard bite to a cross-bite.

Example 27: The dental appliance of any one of examples 1-26, wherein the motion data includes relative motion data on movement of the dental arch relative to an opposing dental arch of the patient.

Example 28: The dental appliance of example 27, wherein the dental arch is a lower arch and the opposing dental arch is an upper arch.

Example 29: The dental appliance of any one of examples 1-28, wherein the appliance is usable for treatment of temporomandibular joint disorders.

Example 30: A method comprising: acquiring an impression of a patient's dentition; acquiring motion data for the patient; generating a thin-shell appliance for the patient based on the impression; generating a contact surface based on the motion data; and joining the contact surface with the thin-shell appliance.

Example 31: The method of example 30, wherein the acquiring an impression includes capturing three-dimensional scan data with an intraoral scanner.

Example 32: The method of any one of examples 30 or 31, wherein the acquiring motion data for the patient includes capturing motion data with a motion capture system.

Example 33: The method of example 32, wherein the capturing motion data with a motion capture system includes: coupling a first clutch to a first dental arch of a patient's dentition; coupling a second clutch to a second dental arch of a patient's dentition; capturing motion data using a first position indicator system of the first clutch; capturing motion data using a second position indicator system of the second clutch; and determining motion of the first dental arch relative to the second dental arch based on the captured motion data from the first position indicator and the second position indicator.

Example 34: The method of any one of examples 31-33, wherein the acquiring motion data for the patient includes acquiring excursive motion data.

Example 35: The method of any one of examples 31-34, wherein the acquiring motion data for the patient includes acquiring protrusive motion data.

Example 36: The method of any one of examples 31-35, wherein the acquiring motion data for the patient includes acquiring extreme lateral motion data.

Example 37: The method of any one of examples 31-36, wherein the generating a thin-shell appliance for the patient based on the impression includes: identifying a portion of a surface of a dental arch in the impression; generating an interior offset surface based on offsetting three-dimensional data representing the identified portion by a first offset amount; generating an exterior offset surface; and joining the interior offset surface to the exterior offset surface.

Example 38: The method of example 37, wherein the generating an exterior offset surface includes generating the exterior offset surface based on offsetting three-dimensional data representing a dental arch from the impression by a second amount.

Example 39: The method of example 37, wherein the generating an exterior offset surface includes generating the exterior offset surface based on offsetting the interior offset surface by a second amount.

Example 40: The method of any one of examples 37-39, wherein the joining the interior offset surface to the exterior offset surface includes: generating an edge surface connecting the interior offset surface to the exterior offset surface.

Example 41: The method of any one of examples 37-40, wherein the identifying the portion of the surface of the dental arch in the impression includes: identifying heights of contour of the dental arch; and extending the portion beyond the heights of contour.

Example 42: The method of any one of examples 30-41, wherein the generating a contact surface based on the motion data includes: generating a contact surface disposed in an anterior region of a dental arch.

Example 43: The method of any one of examples 30-42, wherein the generating a contact surface based on the motion data includes generating a contact surface that has a concave shape in a cross-arch dimension.

Example 44: The method of example 43, wherein the generating a contact surface that has a concave shape in the cross-arch dimension includes sweeping a point from an opposing dental arch through a motion path relative to the dental arch, the motion path being based on the motion data.

Example 45: The method of example 44, wherein the sweeping a point from an opposing dental arch through a motion path includes sweeping the point from an opposing dental arch through a protrusive motion path relative to the dental arch.

Example 46: The method of any one of examples 44 or 45, wherein the generating a contact surface that has a concave shape in the cross-arch dimension includes: moving the opposing dental arch through a hinge motion path in the motion data to position the opposing dental arch in an open position; and sweeping a point of the opposing dental arch in the open position arch through a motion path relative to the dental arch.

Example 47: The method of any one of examples 46, wherein the moving the opposing dental arch through a hinge motion path in the motion data to open the bite by an amount based on a centric relation bite of the patient.

Example 48: The method of any one of examples 43-45, wherein the generating a contact surface that has a concave shape in the cross-arch dimension includes: positioning the opposing dental arch in a centric relation bite position based on the motion data; and sweeping a point of the opposing dental arch in the centric relation bite position arch through a motion path relative to the dental arch.

Example 49: The method of any one of examples 30-48, wherein the contact surface is raised occlusally in an anterior region and slopes away posteriorly.

Example 50: The method of any one of examples 30-49, wherein the contact surface is shaped so that when the thin-shell appliance is worn by the patient, at least a portion of the opposing dental arch remains in contact with the contact surface throughout a motion path from the motion data.

Example 51: The method of example 50, wherein the contact surface is shaped so that when the thin-shell appliance is worn by the patient, at least one cuspid of the opposing dental arch remains in contact with the contact surface throughout a protrusive motion path from the motion data.

Example 52: The method of example 50, wherein the contact surface is shaped so that when the thin-shell appliance is worn by the patient, at least one incisor of the opposing dental arch remains in contact with the contact surface throughout an excursive motion path from the motion data.

Example 53: The method of example 50, wherein the contact surface is shaped so that when the thin-shell appliance is worn by the patient, contact force is balanced approximately evenly across a plurality of teeth of the opposing dental arch.

Example 54: The method of any one of examples 30-53, wherein the generating a contact surface based on the motion data includes generating at least one contact ridge based on the motion data.

Example 55: The method of example 54, wherein the generating at least one contact ridge based on the motion data includes: identifying a contact region of an opposing dental arch; and sweeping the contact region through a motion path relative to the dental arch.

Example 56: The method of example 55, wherein the generating at least one contact ridge based on the motion data further includes deforming a mesh based on the sweeping the contact region.

Example 57: The method of example 55, wherein the generating at least one contact ridge based on the motion data further includes generating a mesh based on the sweeping the contact region; and joining the generated mesh with the contact surface.

Example 58: The method of any one of examples 55-57, wherein: the identifying a contact region of the opposing dental arch includes identifying a contact region on a cuspid of the opposing dental arch; and the sweeping the contact region through the motion path relative to the dental arch includes sweeping the contact region through a protrusive motion path relative to the dental arch.

Example 59: The method of any one of examples 55-58, wherein: the identifying a contact region of the opposing dental arch includes identifying a contact region on an incisor of the opposing dental arch; and the sweeping the contact region through the motion path relative to the dental arch includes sweeping the contact region through an excursive motion path relative to the dental arch.

Example 60: The method of example 59, wherein the generating at least one contact ridge based on the motion data further includes sweeping the contact region through an extreme lateral motion path that corresponds to the opposing dental arch moving form an open bite to a cross bite relative to the dental arch.

Example 61: The method of any one of examples 30-60, wherein the joining the contact surface with the thin-shell appliance includes: generating a solid mesh from the contact surface; and joining the solid mesh to the thin-shell appliance using a Boolean operation.

Example 62: The method of any one of examples 30-60, wherein the joining the contact surface with the thin-shell appliance includes deforming an exterior surface of the thin-shell appliance based on the contact surface.

Example 63: The method of any one of examples 30-62, further comprising: fabricating a physical appliance from the thin-shell appliance using a rapid fabrication machine.

Example 64: The method of example 63, further comprising providing the physical appliance to the patient for treatment of temporomandibular joint disorder.

Example 65: A non-transitory computer-readable storage medium comprising instructions stored thereon that, when executed by at least one processor, cause a computing system to perform the method of any of examples 30-63.

Example 66: A computing device comprising: at least one processor; and memory storing instructions that, when executed by the at least one processor, cause the computing device to perform the method of any of examples 30-63.

Example 67: A system comprising: an intraoral scanner for acquiring an impression of a patient's dentition; a motion capture system for acquiring motion data for the patient; and the computing device of example 66.

Example 68: The system of example 67, further comprising a rapid fabrication machine for fabricating a physical appliance.

Example 69: A dental splint comprising a thin-shell aligner and a contact surface. The contact surface may be formed based on motion data. In some implementations, the motion data includes relative motion data on movement of a patient's upper dentition with respect to the patient's lower dentition. The contact surface may include one or more ridges corresponding to the positions of a contact region on the opposing dentition as a jaw movement is performed. In some examples, the dental splint is used for treatment of temporomandibular joint disorder.

Example 70: A method comprising: acquiring an impression of a patient's dentition; acquiring jaw motion data for the patient; generating a thin-shell appliance for the patient based on the impression; generating a contact surface based on the motion data; and joining the contact surface with the thin-shell appliance.

1. A dental appliance for a dental arch of a patient, the appliance comprising:
an interior surface shaped to fit to contours of at least one tooth of the dental arch; and
an exterior surface that includes at least one contact surface formed based on motion data.

2. The dental appliance of claim 1, wherein the exterior surface includes a contour-following surface that follows the contours of at least one tooth of the dental arch, wherein the contour-following surface is an offset surface of the interior surface.

3. (canceled)

4. The dental appliance of claim 1, wherein the contact surface is disposed in an anterior region of the dental arch.

5. (canceled)

6. (canceled)

7. (canceled)

8. The dental appliance of claim 1, wherein the contact surface has a shape that corresponds to a shape formed by sweeping a point from an opposing dental arch through a motion path relative to the dental arch, the motion path being based on the motion data.

9. (canceled)

10. (canceled)

11. The dental appliance of claim 1, wherein the contact surface is offset in an occlusal direction by a predetermined amount, wherein the predetermined amount is based on a centric relation bite of the patient.

12. (canceled)

13. (canceled)

14. (canceled)

15. The dental appliance of claim 1, wherein the contact surface is shaped so that when the dental appliance is worn by the patient, at least one cuspid of an opposing dental arch of the patient remains in contact with the contact surface throughout a protrusive motion path from the motion data.

16. The dental appliance of claim 1, wherein the contact surface is shaped so that when the dental appliance is worn by the patient, at least one anterior tooth of an opposing dental arch of the patient remains in contact with the contact surface throughout an excursive motion path from the motion data.

17. (canceled)

18. (canceled)

19. (canceled)

20. (canceled)

21. (canceled)

22. (canceled)

23. (canceled)

24. (canceled)

25. (canceled)

26. (canceled)

27. The dental appliance of claim 1, wherein the motion data includes relative motion data on movement of the dental arch relative to an opposing dental arch of the patient.

28. (canceled)

29. (canceled)

30. A method comprising:
acquiring an impression of a patient's dentition based on at least one of (i) capturing three-dimensional scan data with an intraoral scanner and (ii) capturing motion data with a motion capture system;
acquiring motion data for the patient that includes at least one of (i) excursive motion data, (ii) protrusive motion data, and (iii) extreme lateral motion data;
generating a thin-shell appliance for the patient based on the impression;
generating a contact surface based on the motion data; and
joining the contact surface with the thin-shell appliance.

31. (canceled)

32. (canceled)

33. The method of claim 30, wherein the capturing motion data with a motion capture system includes:
coupling a first clutch to a first dental arch of a patient's dentition;
coupling a second clutch to a second dental arch of a patient's dentition;
capturing motion data using a first position indicator system of the first clutch;
capturing motion data using a second position indicator system of the second clutch; and
determining motion of the first dental arch relative to the second dental arch based on the captured motion data from the first position indicator and the second position indicator.

34. (canceled)

35. (canceled)

36. (canceled)

37. The method of claim 30, wherein the generating a thin-shell appliance for the patient based on the impression includes:
identifying a portion of a surface of a dental arch in the impression based at least in part on identifying heights of contour of the dental arch and extending the portion beyond the heights of contour;
generating an interior offset surface based on offsetting three-dimensional data representing the identified portion by a first offset amount;
generating an exterior offset surface based on at least one of (i) offsetting three-dimensional data representing a dental arch from the impression by a second amount and (ii) offsetting the interior offset surface by a second amount; and
joining the interior offset surface to the exterior offset surface based on generating an edge surface connecting the interior offset surface to the exterior offset surface.

38. (canceled)

39. (canceled)

40. (canceled)

41. (canceled)

42. The method of claim 30, wherein the generating a contact surface based on the motion data includes:
generating a contact surface disposed in an anterior region of a dental arch, and
generating a contact surface that has a concave shape in a cross-arch dimension based on sweeping a point from an opposing dental arch through a motion path relative to the dental arch, the motion path being based on the motion data.

43. (canceled)

44. (canceled)

45. (canceled)

46. The method of claim 42, wherein the generating a contact surface that has a concave shape in the cross-arch dimension includes:
moving the opposing dental arch through a hinge motion path in the motion data to position the opposing dental arch in an open position based at least in part on a centric relation bite of the patient; and
sweeping a point of the opposing dental arch in the open position arch through a motion path relative to the dental arch.

47. (canceled)

48. (canceled)

49. (canceled)

50. (canceled)

51. (canceled)

52. (canceled)

53. (canceled)

54. The method of claim 30, wherein the generating a contact surface based on the motion data includes generating at least one contact ridge based on the motion data by identifying a contact region of an opposing dental arch and sweeping the contact region through a motion path relative to the dental arch.

55. (canceled)

56. The method of claim 54, wherein the generating at least one contact ridge based on the motion data further includes deforming a mesh based on the sweeping the contact region.

57. The method of claim 54, wherein the generating at least one contact ridge based on the motion data further includes
generating a mesh based on the sweeping the contact region; and
joining the generated mesh with the contact surface.

58. The method of claim 54, wherein:
the identifying a contact region of the opposing dental arch includes identifying a contact region on a cuspid of the opposing dental arch; and
the sweeping the contact region through the motion path relative to the dental arch includes sweeping the contact region through a protrusive motion path relative to the dental arch.

59. The method of claim 54, wherein:
the identifying a contact region of the opposing dental arch includes identifying a contact region on an incisor of the opposing dental arch; and
the sweeping the contact region through the motion path relative to the dental arch includes sweeping the contact region through an excursive motion path relative to the dental arch.

60. The method of claim 59, wherein the generating at least one contact ridge based on the motion data further includes sweeping the contact region through an extreme lateral motion path that corresponds to the opposing dental arch moving form an open bite to a cross bite relative to the dental arch.

61. (canceled)

62. (canceled)

63. (canceled)

64. (canceled)

65. (canceled)

66. (canceled)

67. (canceled)

68. (canceled)
